(19) United States
(12) Patent Application Publication
(10) Pub. No.: US 2025/0236897 A1
(43) Pub. Date: Jul. 24, 2025

(54) PROCESSES FOR PREPARATION OF AVACOPAN AND INTERMEDIATES THEREOF

(71) Applicant: TEVA PHARMACEUTICALS INTERNATIONAL GMBH, Jona (CH)

(72) Inventors: Imre Gyujto, Debrecen (HU); Tihamer Paal, Debrecen (HU); Zsuzsa Potarine Juhasz, Debrecen (HU); Daniel Eszenyi, Tiszalok (HU); Gabor Jozsef Zahuczky, Bocskaikert (HU); Zoltan Kondor, Kocsord (HU); Vadivelan Rengasamy, Ariyalur District (IN); Ramkaran Prajapati, Greater Noida (IN); Vaibhav Suresh Daund, Nashik (IN); Shachi Sainger, Ghaziabad (IN); Rushikesh Kadu, Ahmedabad (IN); Vishal Sharma, Yamunanagar (IN)

(21) Appl. No.: 18/835,478

(22) PCT Filed: Feb. 16, 2023
(86) PCT No.: PCT/US2023/013189

§ 371 Date: Aug. 2, 2024

Related U.S. Application Data
(60) Provisional application No. 63/439,309, filed on Jan. 17, 2023.
(60) Provisional application No. 63/330,799, filed on Apr. 14, 2022.

(30) Foreign Application Priority Data
Feb. 16, 2022 (IN) 202211008251
Apr. 8, 2022 (IN) 202211021098
Nov. 24, 2022 (IN) 202211067518

(51) Int. Cl. C12P 17/12; C07D 211/60; C07D 211/78; C12N 9/04

(52) U.S. Cl. CPC C12P 17/12; C07D 211/60; C07D 211/78; C12N 9/0006; C12Y 101/01001; C12Y 101/01047

(57) ABSTRACT

The present disclosure encompasses a process for chiral resolution of racemic amines, particularly ethyl 2-(4-((tert-butoxycarbonyl)amino)phenyl)piperidine-3-carboxylate, into its desired isomer, ethyl (2R,3S)-2-(4-((tert-butoxycarbonyl)amino)phenyl)-piperidine-3-carboxylate, which is an intermediate useful in the synthesis of Avacopan.

### FIELD OF THE DISCLOSURE

The present disclosure encompasses process for preparing Avacopan, particularly to an optical resolution of racemic ethyl 2-(4-((tert-butoxycarbonyl)amino)phenyl)piperidine-3-carboxylate, an intermediate in the synthesis of Avacopan, into its desired isomer, ethyl (2R,3S)-2-(4-((tert-butoxycarbonyl)amino)phenyl)piperidine-3-carboxylate.

The present disclosure also encompasses kinetic resolution of cyclic amine compounds, using imine reductase enzymes. The present disclosure encompasses a process for preparing Avacopan, particularly to a kinetic resolution of racemic cis-ethyl 2-(4-((tert-butoxycarbonyl)amino)phenyl)piperidine-3-carboxylate, an intermediate in the synthesis of Avacopan, into its desired isomer, ethyl (2R,3S)-2-(4-((tert-butoxycarbonyl)amino)phenyl)-piperidine-3-carboxylate.

### BACKGROUND OF THE DISCLOSURE

Avacopan chemical name is (2R,3S)-2-[4-(cyclopentylamino)phenyl]-1-(2-fluoro 6-methylbenzoyl)-N-[4-methyl-3-(trifluoromethyl)phenyl]piperidine-3 carboxamide, having the following chemical structure:

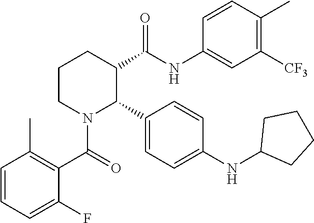

Avacopan is a Complement C5a receptor antagonist, approved for the treatment of ANCA-associated Vasculitis.

The compound is described in PCT publication WO 2010/075257. In this process, ethyl-2-(4-((tert-butoxycarbonyl)amino)phenyl)piperidine-3-carboxylate, a racemic intermediate is optically resolved into the desired enantiomer, ethyl (2R,3S)-2-(4-((tert-butoxycarbonyl)amino)phenyl)piperidine-3-carboxylate, by crystallization of L-Tolyl tartaric acid (L-DTTA) salt, in a 14.1% yield.

PCT publication WO 2016/053890 relates to two synthetic processes for preparation of Avacopan and to intermediates. In these processes, the enantiomeric separation is done via L-DTTA salt in an overall yield of 20.2%.

Imine reductase enzymes (IREDs) are typically NADPH-dependent enzymes utilized for the asymmetric reduction of cyclic imines such as, pyrrolidines, piperidines and 3,4-dihydroisoquinolines. In addition, IREDs have been shown to have reductive aminase (RedAm) activity as well, leading to their use as biocatalysts for reductive aminations.

France, et al. (ACS Catal. 2016, 6, 3753-3759) describes a biocatalytic cascade involving carboxylic acid reductase, ω-transaminase and imine reductase enzymes to synthesize one-pot (2R,3S)-3-methyl-2-phenylpiperidine starting from 4-methyl-5-oxo-5-phenylpentanoic acid.

There is a need for additional process for preparing Avacopan in a high chemical and enantiomeric purity, and a high yield that can be utilized in large-scale commercial operations.

### SUMMARY OF THE DISCLOSURE

In one aspect, the present disclosure is based on the surprising discovery that imine reductase enzymes are able to provide stereochemical control in the resolution of two vicinal stereogenic centres in a cyclic amine system through enantioselective oxidation of the cyclic amine. In particular it has been unexpectedly found that by reversing their usual mode of action, oxidative kinetic resolution of racemic-cis-2,3-substituted piperidines can be effected, wherein one enantiomer is oxidized, and the other is unreacted. This selectivity of imine reductases operating in this mode has been utilized in the optical resolution of a racemic cis-piperidine intermediate, cis-ethyl 2-(4-((tert-butoxycarbonyl)-amino)phenyl)-piperidine-3-carboxylate (Compound 4) of Avacopan. Advantageously, the imine reductase enzyme system is able to leave the desired 2R,3S enantiomer untouched, whilst selectively oxidizing the undesired enantiomer to the imine and its more stable enamine tautomer. Typically, the enamine tautomer predominates. This enantiomeric selectively has been exploited by the present inventors to provide an efficient process for the separation of the desired 2R,3S enantiomer, as well as recovery and recycling of the undesired enantiomer back to the racemic Compound 4 through an ex situ racemization process, thereby providing increased yields and purity of the desired 2R,3S enantiomer of Compound 4, particularly useful in the synthesis of Avacopan.

The present disclosure encompasses processes for preparing Avacopan, particularly to a kinetic resolution of cis-ethyl 2-(4-((tert-butoxycarbonyl)amino)phenyl)-piperidine-3-carboxylate (Compound 4) to ethyl (2R,3 S)-2-(4-((tert-butoxycarbonyl)amino)-phenyl)piperidine-3-carboxylate (Compound 5). In this process, an enamine compound, ethyl 2-(4-((tert-butoxycarbonyl)amino)phenyl)-1,4,5,6-tetrahydropyridine-3-carboxylate (“Compound 5A”) is also formed, and can be efficiently recycled. The process involves kinetic resolution of the cyclic amine moiety, and optionally recycling of Compound 5A back into Compound 4. The process may utilize a cofactor regeneration system, such as NADPH oxidase (NOX) or alcohol dehydrogenase (ADH) or ketoreductase (KRED), particularly alcohol dehydrogenase.

In one embodiment, the present disclosure encompasses a process for preparing Compound 5 in high enantiomeric purity and high yield.

In one embodiment, the present disclosure encompasses a process for preparing Avacopan, comprising preparing Compound 5 in high enantiomeric purity and high yield.

In one embodiment, the present disclosure encompasses a kinetic resolution of cyclic amines, comprising subjecting cyclic amine to imine reductase enzymes.

In a further embodiment, the present disclosure encompasses a kinetic resolution of cyclic amines, comprising subjecting a cyclic amine to imine reductase enzymes in the presence of NOX or ADH or KRED as a cofactor regeneration system.

In a specific embodiment, the present disclosure encompasses a kinetic resolution of cyclic amines, which is followed by ex-situ recycling of the side-formed imine/enamine compound back into the starting cyclic amine compound.

In one embodiment, the present disclosure encompasses a kinetic resolution of Compound 4, comprising subjecting Compound 4 to imine reductase enzymes.

In a further embodiment, the present disclosure encompasses a kinetic resolution of Compound 4, comprising subjecting Compound 4 to imine reductase enzymes in the presence of NOX or ADH or KRED as a cofactor regeneration system. The cofactor regenerating system can contain NADPH, NADP+ or their mixture as cofactor. Optionally the cofactor regenerating system comprises: a cofactor regenerating enzyme, such as an NADPH oxidase (NOX), an alcohol dehydrogenase (ADH) or a ketoreductase (KRED), preferably wherein the cofactor regenerating system comprises ADH or KRED wherein acetone is used as sacrificial substrate, most preferably the cofactor regenerating enzyme comprises ADH from Lactobacillus brevis.

In a specific embodiment, the present disclosure encompasses a kinetic resolution of Compound 4, which is followed by ex-situ recycling of Compound 5A back into the starting Compound 4.

In one embodiment, the present disclosure encompasses a process for preparing Avacopan, comprising a kinetic resolution, as discussed herein.

The present disclosure further encompasses process for preparing Avacopan, particularly to an optical resolution of Compound 4 and recycling of the isomer ethyl (2S,3R)-2-(4-((tert-butoxycarbonyl)amino)phenyl)piperidine-3-carboxylate (“Compound 5-2S,3R”).

In one embodiment, the present disclosure encompasses a process for preparing Compound 5 in high enantiomeric purity and high yield.

In one embodiment, the present disclosure encompasses a process for preparing Avacopan, comprising preparing Compound 5 in high enantiomeric purity and high yield.

In some embodiments, the present disclosure encompasses Compound 4-Cl, and its use in preparation of Avacopan.

In yet another embodiment, the present disclosure encompasses a process provides alternative processes for preparation of Avacopan, without using the pyrophoric reagent trimethyl aluminum; as described herein below.

### BRIEF DESCRIPTION OF THE DRAWINGS

FIG. 1 shows a PXRD pattern of Amorphous Avacopan.

FIG. 2 sets forth amino acid sequences of IRED enzymes used in accordance with the present disclosure.

### DETAILED DESCRIPTION OF THE DISCLOSURE

As used herein, ethyl 2-(4-((tert-butoxycarbonyl)amino)phenyl)-nicotinate is referred to as “Compound 3”.

As used herein, the racemic compound ethyl 2-(4-((tert-butoxy-carbonyl)amino)phenyl)piperidine-3-carboxylate is referred to as “Compound 4”. Preferably, Compound 4 is in its cis form, i.e. cis-ethyl 2-(4-((tert-butoxycarbonyl)amino)phenyl)piperidine-3-carboxylate.

As used herein, the compound ethyl (2R,3S)-2-(4-((tert-butoxy-carbonyl)amino)phenyl)piperidine-3-carboxylate is referred to as “Compound 5”.

As used herein, the compound ethyl (2S,3R)-2-(4-((tert-butoxycarbonyl)amino)phenyl)piperidine-3-carboxylate is referred to as “Compound 5-ML” or “Compound 5-2S, 3R”.

As used herein, the compound ethyl (2S,3R)-2-(4-((tert-butoxy-carbonyl)amino)phenyl)-1-chloropiperidine-3-carboxylate is referred to as “Compound 4-Cl”.

As used herein, the compound ethyl 2-(4-((tert-butoxycarbonyl)-amino)phenyl)-1,4,5,6-tetrahydropyridine-3-carboxylate is referred to as “Compound 5A”.

As used herein, the compound ethyl 2-(4-((tert-butoxycarbonyl)-amino)phenyl)-3,4,5,6-tetrahydropyridine-3-carboxylate or ethyl (R)-2-(4-((tert-butoxycarbonyl)amino)phenyl)-3,4,5,6-tetrahydropyridine-3-carboxylate is referred to as “Compound 5B”.

As used herein, the compound ethyl (S)-2-(4-((tert-butoxycarbonyl)amino)phenyl)-3,4,5,6-tetrahydropyridine-3-carboxylate is referred to as “Compound 5D”.

As used herein, the compound ethyl (2R,3S)-2-(4-((tert-butoxy-carbonyl)amino)phenyl)-1-(2-fluoro-6-methylbenzoyl)piperidine-3-carboxylate, referred to as “Compound 7”

As used herein, the compound, ethyl (2R,3S)-2-(4-aminophenyl)-1-(2-fluoro-6-methylbenzoyl)piperidine-3-carboxylate, referred to as “Compound 8”

As used herein, the compound, (2R,3S)-2-(4-aminophenyl)-1-(2-fluoro-6-methylbenzoyl)piperidine-3-carboxylic acid, referred to as “Compound 8-OH”

As used herein, the compound, ethyl (2R,3S)-2-(4-(Cyclopentyl-amino)phenyl)-1-(2-fluoro-6-methylbenzoyl)piperidine-3-carboxylate, referred to as “Compound 10”

As used herein, the compound, (2R,3S)-2-(4-(cyclopentylamino)-phenyl)-1-(2-fluoro-6-methylbenzoyl)piperidine-3-carboxylic acid, referred to as “Compound 10-OH”.

As used herein, the compound, 2-(4-((tert-butoxycarbonyl)-amino)phenyl)nicotinic acid, referred to as “Compound 29”.

As used herein, the compound, tert-butyl (4-(3-((4-methyl-3-(trifluoromethyl)phenyl)carbamoyl) pyridin-2-yl)phenyl)carbamate, referred to as “Compound 30”.

As used herein, the compound, tert-butyl(4-(3-((4-methyl-3-(trifluoromethyl)phenyl)carbamoyl) piperidin-2-yl)phenyl)carbamate, referred to as “Compound 31”.

As used herein, the compound, tert-butyl (4-((2R,3S)-3-((4-methyl-3-(trifluoromethyl)phenyl) carbamoyl)piperidin-2-yl)phenyl)carbamate, referred to as “Compound 32”.

As used herein, HATU refers to hexafluorophosphate azabenzotriazole tetramethyl uranium, or 1-[bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide.

As used herein, the term “isolated” in reference to a compound of the present disclosure, such as Avacopan or Avacopan intermediate, corresponds to solid state form of a compound, such as Avacopan or Avacopan intermediate that is physically separated from the reaction mixture in which it is formed.

As used herein, the term “ex-situ recycling” in reference to Compound 5A of the present disclosure, refers to a separate step of isolation of Compound 5A from the reaction mixture employed in the process of the present disclosure, and further converting it to the racemate form, i.e. Compound 4.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of from about 10 mbar to 50 mbar.

As used herein and unless indicated otherwise, the term “ambient conditions” refer to atmospheric pressure and a temperature of about 22-24° C.

As used herein, the term “cofactor regeneration system” means an enzyme-substrate pair, mixed to the required constituents of the enzymatic reaction (including the cofactor). In the course of imine reductase catalyzed oxidation, cofactor regeneration system has the role to oxidize the reduced form of the β-nicotinamide adenine dinucleotide phosphate cofactor (NADPH) to the oxidized form (NADP+), therefore allowing the use of catalytic amount of NADP+. It is generally understood that the cofactor regeneration system is named after the enzyme that is used for the oxidation of NADPH to NADP+. Typically, the name implicates the suitable coupled substrate. For example, the NOX cofactor regeneration system implicates the use of molecular oxygen (O₂) as a substrate together with NOX enzyme, whereas the ADH or KRED cofactor regeneration systems implicate the use of a carbonyl compound as substrate. In case of imine reductase catalyzed reduction, cofactor regeneration system has the role to reduce the oxidized form of the 0-nicotinamide adenine dinucleotide phosphate cofactor (NADP+) to the reduced form (NADPH), therefore allowing the use of catalytic amount of NADP+. GDH cofactor regeneration system implicates the use of glucose as a substrate together with GDH enzyme.

As used herein, reference to an amino acid sequence have a specified percentage identity to a specified amino acid sequence refers to the degree of similarity between the two amino acid sequences. The percentage may be determined by comparing with the naked eye or using a bioinformatic algorithm. The latter enables calculation of the degree of homology by aligning sequences for comparison. The homology between the two amino acid sequences may be calculated as a percentage. The useful automated algorithms may be used in GAP, BESTFIT, FASTA, and TFASTA computer software modules of Wisconsin Genetics Software Package (Genetics Computer Group, Madison, Wis., USA). Other useful algorithms and homology determinations on alignment are already automated in software such as FASTP, BLAST, BLAST2, PSIBLAST, and CLUSTAL W. Preferably, CLUSTAL Omega (https://www.uniprot.org/align) was used for the sequence identity calculations.

### Process for Kinetic Resolution of Cyclic Amines and Use in the Synthesis of Avacopan

In one aspect, the present disclosure provides a process for kinetic resolution of a racemic amine, particularly racemic 2,3-disubstituted piperidine, by enzymatic oxidation to an imine or to an enamine or their mixture, comprising reacting the racemic 2,3-disubstituted piperidine with an imine reductase enzyme to form a mixture comprising enriched enantiomer of the amine, an imine and/or enamine. Surprisingly, the imine reductase enzyme can selectively oxidize one isomer of the racemic amine to form an imine and/or enamine. The other isomer remains unreacted. The process therefore provides for enrichment of one isomer of the amine. Advantageously, the process can further comprise the recovery of enamine resulting from the oxidation of the undesired enantiomer, which is of further use as explained below.

Preferably, the kinetic resolution comprises:
(a) reacting a racemic amine having the formula:

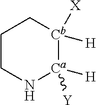

wherein X is alkyl, aryl or functional group, preferably wherein the functional group is an electron withdrawing group, and Y is H, C₁ to C₈ alkyl group, or aryl group, preferably a substituted phenyl, optionally wherein the substituent is selected from 4-amino, 4-t-butoxycarbonylamino, and 4-cyclopentylamino (i.e. optionally wherein Y is 4-aminophenyl, 4-t-butoxycarbonylaminophenyl or 5-cyclopentylaminophenyl:

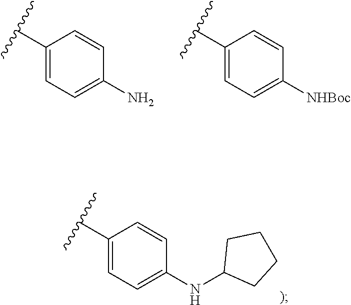 [SMILES: *c1ccc(N)cc1 ; *c1ccc(NC(=O)OC(C)(C)C)cc1 ; *c1ccc(NC2CCCC2)cc1]

wherein one or both of C^{a} and C^{b} is a chiral center, to form a mixture comprising the chiral 2,3-disubstituted piperidine, imine, and enamine:

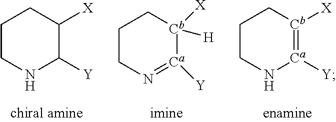

(b) optionally isolating the chiral amine; and
(c) optionally isolating the imine and/or enamine and converting the imine or enamine to the racemic amine.

X and Y are preferably in a cis configuration. Particularly, the racemic amine has the formula:

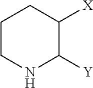

wherein X is C₁ to C₈ alkyl, C₆ to C₁₀ aryl or functional group, preferably an electron withdrawing group, particularly (C₁ to C₁₀ alkoxy)carbonyl, carboxyl, carbamoyl, cyano, formyl, C₁ to C₈ acyl, most preferably (C₁ to C₁₀ alkoxy)carbonyl; and Y is H, C₁ to C₈ alkyl, or an optionally substituted C₆ to C₁₀ aryl, preferably a substituted phenyl, optionally wherein the substituent is selected from 4-amino, 4-t-butoxycarbonylamino, and 4-cyclopentylamino. The racemic amine can have the formula:

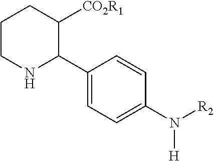

wherein: R₁ is a C₁ to C₈ alkyl group, a or a C₁ to C₆ alkyl group; and R₂ is H, C₁ to C₆ alkyl group, a C₅ to C₈ cycloalkyl group, or a protecting group, particularly wherein R₂ is cyclopentyl or t-butyloxycarbonyl (Boc), and more particularly Boc. Preferably R₁ is a C₁ to C₃ alkyl group, and preferably R₁ is ethyl, more preferably wherein R₁ is ethyl and R₂ is Boc. Preferably, the racemic amine is Compound 4 having the formula:

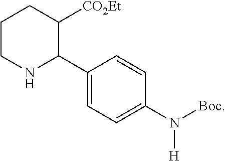 [SMILES: CCOC(=O)C1CCCNC1c1ccc(NC(=O)OC(C)(C)C)cc1]

Preferably, the chiral amine is the desired isomer, and which is unreacted. The imine reductase may advantageously selectively oxidize the undesired isomer of the racemic amine to form an imine and/or enamine. The imine and enamine may be a tautomeric mixture:

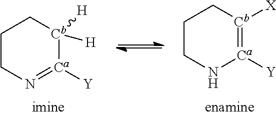

Typically, the enamine form may predominate.

According to any embodiment, the reaction is preferably carried out in the presence of a cofactor regenerating system containing NADPH, NADP+ or their mixture as cofactor, optionally wherein the cofactor regenerating system comprises: cofactor regenerating enzyme, such as an NADPH oxidase (NOX), an alcohol dehydrogenase (ADH) or a ketoreductase (KRED), and preferably wherein the cofactor regenerating system comprises ADH or KRED wherein acetone is used as sacrificial substrate, most preferably comprises ADH from Lactobacillus brevis.

According to any embodiment, the reaction may be carried out in a suitable buffer, such as a TRIS buffer, or phosphate buffer, at a suitable pH, for example about 7.8 to about 8.8, about 8.0 to about 8.6, or about 8.5; a phosphate buffer such as KH2PO4 buffer at a suitable pH, for example about 7.0 to about 8.0, about 7.2 to about 7.8, or about 7.5; or potassium phosphate buffer at a suitable pH, for example about 7.0 to about 8.0, about 7.2 to about 7.8, or about 7.5.

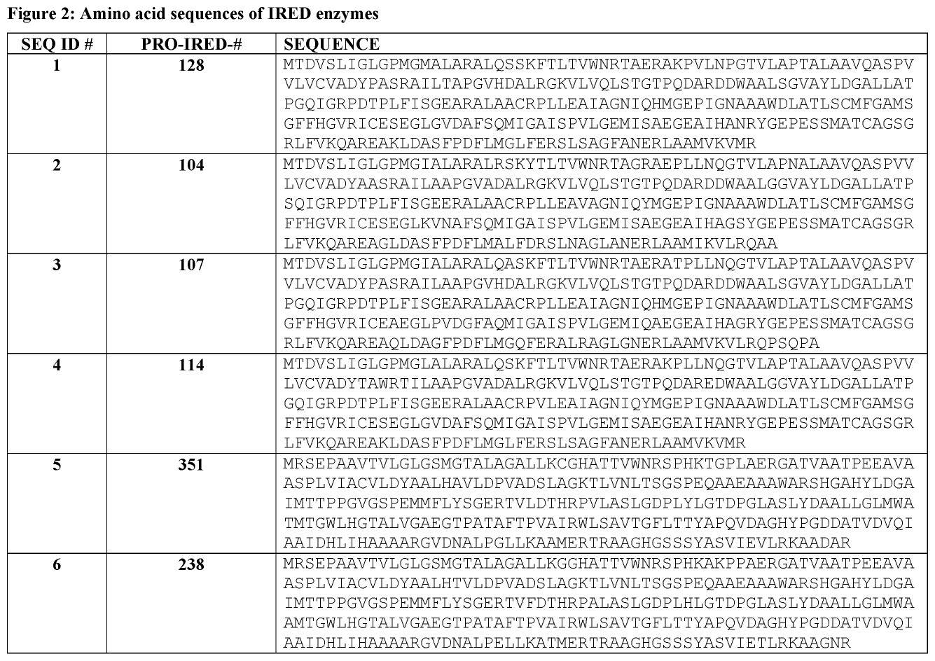

Suitable imine reductase enzymes include those related to Variovorax sp. such as Variovorax_beijingensis. Especially suitable imine reductase enzymes are those having an amino acid sequence that comprises: at least 80%, at least 85%, at least 90%, at least 92%, at least 95%, at least 96%, at least 97%, at least 98%, and at least 99% identity to SEQ ID 1 (FIG. 2, PRO-IRED-128, which is available commercially from Prozomix Limited). Particularly preferred imine reductase enzymes have amino acid sequences corresponding to SEQ ID 1, SEQ ID 2, SEQ ID3 and SEQ ID 4 (FIG. 2), which are commercially available from Prozomix Limited as PRO-IRED-128, PRO-IRED-104, PRO-IRED-107 and PRO-IRED-114. Imine reductases were explored by Prozomix, and were of found in soil sample in the United Kingdom (Marshall et al. Nat. Chem. 2021 13, 140-148). Prozomix imine reductase termed as pIR, pIRED in the above cited literature refer to identical enzymes as the PRO-IRED coding if the respective number codes coincide (e.g. pIR128 and pIRED128 is identical with PRO-IRED-128). Imine reductases can be clustered to superfamilies (SFam1) according to Imine Reductase Engineering Database (Fademrech et al. Proteins 2016, 84 (5), 600-610; https://ired.biocatnet.de/).

Advantageously the kinetic enzymatic resolution process described herein, results in the oxidation of the undesired isomer of the racemic amine, to form the corresponding imine and/or enamine, preferably as a tautomeric mixture, wherein the enamine predominates. Preferably, the imine/enamine may be separated from the unreacted enantiomer, by filtering off the imine/enamine as a solid or optionally by extracting the imine/enamine at acidic pH. Preferably, the desired isomer is unreacted, and can be recovered from the reaction mixture by extraction once the imine/enamine is filtered off, or optionally by pH dependent extraction at pH 8.2. The recovered enamine can be readily isolated and recycled ex situ back to the racemic amine. In particular, the enamine can be subjected to catalytic hydrogenation to convert the enamine back to the racemic amine. The process may be repeated to generate further yield of the desired isomer of the amine. Preferably the racemic amine is cis-ethyl 2-(4-((tert-butoxycarbonyl)-amino)phenyl)-piperidine-3-carboxylate (Compound 4), which is a useful intermediate for the preparation of Avacopan. The utility of the imine reductase kinetic resolution in the synthesis of Avacopan is described in further detail below.

In another aspect, the present disclosure encompasses a process for preparing Avacopan, particularly to a kinetic resolution of cis-ethyl 2-(4-((tert-butoxycarbonyl)amino)phenyl)-piperidine-3-carboxylate (Compound 4) to ethyl (2R,3S)-2-(4-((tert-butoxycarbonyl)amino)-phenyl)piperidine-3-carboxylate (Compound 5). In this process, an enamine compound, ethyl 2-(4-((tert-butoxycarbonyl)amino)phenyl)-1,4,5,6-tetrahydropyridine-3-carboxylate (“Compound 5A”) is also formed, and can be efficiently recycled. The process involves kinetic resolution of the cyclic amine moiety, and optionally recycling of Compound 5A back into Compound 4. The process may utilize a cofactor regeneration system, such as NOX or ADH or KRED.

In this process, the kinetic resolution is performed by an imine reductase enzyme, which, surprisingly, enantioselectively oxidizes one enantiomer of the cyclic amine compound, and the desired enantiomer remains in the reaction mixture and may be further used in the reaction. The oxidized (thereafter tautomerized form) product may be simply and efficiently isolated and further recycled into the starting compound, Compound 4, which makes the process highly efficient and desired for large scale production.

Additionally, the process can be done by using ADH or KRED cofactor regeneration system, which is a robust cofactor regenerating enzyme, that affords employing a simple and low-cost co-substrate, like acetone, as a hydride acceptor. In addition, the ADH or KRED cofactor regeneration system affords conducting the kinetic resolution using catalytic amount of nicotinamide adenine dinucleotide phosphate (NADP+) cofactor.

In this process, enamine compound, Compound 5A, which is also formed as a by-product, may be further recycled from the reaction mixture via extraction at acidic pH, or by precipitation and filtering, or by filtering from the reaction mixture, and optionally crystallising.

The present disclosure encompasses a process for kinetic resolution of a cyclic amine compound by an imine reductase enzyme. This process may further comprise recycling of the side-formed enamine compound, back into racemic form of the starting cyclic amine compound. This recycling may be done ex-situ, in high yield and purity.

The above process may comprise the utilization of a cofactor regeneration system, such as NADPH oxidase (NOX) or alcohol dehydrogenase (ADH) or ketoreductase (KRED). In a specific embodiment, the present disclosure comprises a process for kinetic resolution of a cyclic amine compound by an imine reductase enzyme and ADH cofactor regenerating system.

The above described kinetic resolution may be used in a process for preparing Avacopan. Particularly, this process may be applied for increasing the enantiomeric purity of Avacopan intermediate, Compound 5, in a high yield.

The present disclosure encompasses a process for kinetic resolution of a Compound 4 by an imine reductase enzyme, to obtain Compound 5, which may further used to prepare Avacopan. This process may further comprise recycling of the side-formed enamine compound, Compound 5A, back into Compound 4. This recycling may be done ex-situ, in high yield and purity.

The above process may comprise the utilization of a cofactor regeneration system, such as NADPH oxidase (NOX) or alcohol dehydrogenase (ADH) or ketoreductase (KRED). In a specific embodiment, the present disclosure comprises a process for kinetic resolution of a cyclic amine by an imine reductase enzyme and ADH/KRED cofactor regenerating system.

In the process of the present disclosure, Compound 4 is first subjected to kinetic resolution to obtain Compound 5, which is further reacted to obtain Avacopan. The enamine compound, Compound 5A, is further recycled from the reaction mixture.

The above described process can be illustrated by the following Scheme 1:

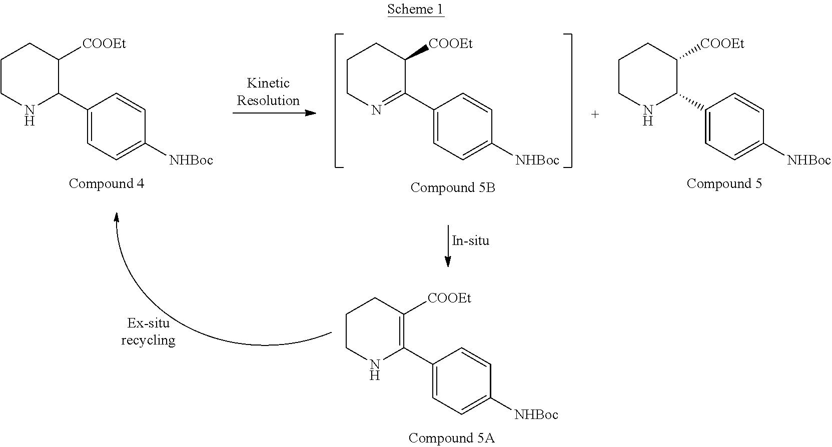

The above described process can provide the desired compound 5 in high quality, and may be efficiently used in high-scale.

The isolation of Compound 5A may be performed by acid-base extraction. Alternatively, Compound 5A may be crystallized and filtered, or filtered out from reaction mixture, therefore providing a solution comprising Compound 5, which can be converted to Avacopan in subsequent reaction steps, the filtered Compound 5A may be recycled to Compound 4.

In specific embodiments, the present disclosure encompasses a process for recycling Compound 5A to Compound 4, comprising crystallizing Compound 5A from a reaction mixture comprising Compound 5 and Compound 5A, isolating the crystallized Compound 5A and further converting it to Compound 4. The remaining Compound 5 may be used to prepare Avacopan.

Compound 5A may be converted back to Compound 4. This conversion can be done by hydrogenation using a catalyst, for example Pd/C, Pt₂O or Raney nickel in acetic acid.

Alternatively, the recovered Compound 5A can be converted to Compound 5 by dynamic kinetic reduction.

In specific embodiments, the present disclosure encompasses a process for preparing Compound 5 comprising reducing Compound 5A using imine reductase enzyme and glucose dehydrogenase (GDH) as a cofactor regeneration system. Preferably, the cofactor regenerating system, may contain NADPH, NADP+ or their mixture as cofactor, optionally wherein the cofactor regenerating enzyme is glucose dehydrogenase (GDH).

Suitable imine reductase enzymes include those related to Streptomyces sp. (such SEQ ID 5 from Streptomyces albus; and SEQ ID 6 from Streptomyces rimosus). Especially suitable imine reductase enzymes are those having an amino acid sequence that comprises: at least 80%, at least 85%, at least 90%, at least 92%, at least 95%, at least 96%, at least 97%, at least 98%, and at least 99% identity to SEQ ID 5 (FIG. 2, PRO-IRED-351, which is available commercially from Prozomix Limited). Particularly preferred imine reductase enzymes have amino acid sequences corresponding to SEQ ID 5 and SEQ ID 6 (FIG. 2), which are commercially available from Prozomix Limited as PRO-IRED-351 and PRO-IRED-238).

This process can provide Compound 5 in high diastereomeric and enantiomeric purity. Typically, in this process, Compound 5D is formed as an intermediate which is converted to Compound 5. This process may be illustrated by the following Scheme 2:

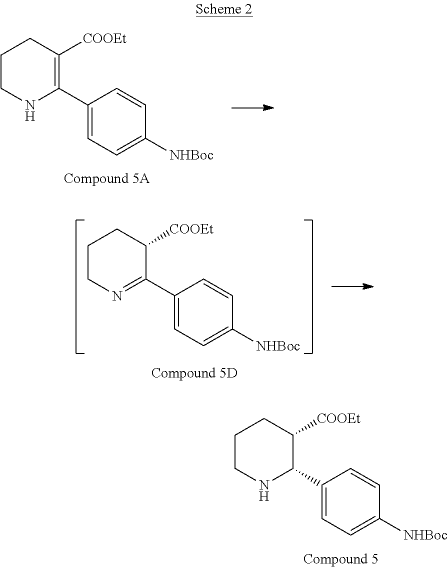

Compound 5 is used further to prepare Avacopan. The preparation of Avacopan can be done by any method disclosed in the literature, for example as described in WO 2010/075257.

The present disclosure encompasses a process for preparing imine reductase enzyme, referred to herein as TPW-T164; and a process for preparing alcohol dehydrogenase enzyme, referred to herein TPW-T004. The processes are described in the examples herein below.

### Process for Optical Resolution of Compound 4 and its Use in the Synthesis of Avacopan

The present disclosure further provides a process for the optical resolution of racemic ethyl 2-(4-((tert-butoxycarbonyl)-amino)phenyl)piperidine-3-carboxylate (Compound 4):

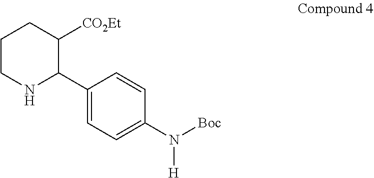 [SMILES: CCOC(=O)C1CCCNC1c1ccc(NC(=O)OC(C)(C)C)cc1]

comprising:
(a) reacting Compound 4 with L-tolyl tartaric acid (L-DTTA);
(b) separating the L-DTTA salt of ethyl (2R,3 S)-2-(4-((tert-butoxycarbonyl)amino)phenyl)piperidine-3-carboxylate (Compound 5 L-DTTA salt) from the L-DTTA salt of ethyl (2S,3R)-2-(4-((tert-butoxycarbonyl)amino)phenyl)piperidine-3-carboxylate (Compound 5-ML L-DTTA salt); and
(c) recycling Compound 5 ML L-DTTA salt by converting to racemic Compound 4.

Preferably Compound 5 L-DTTA salt is isolated from the reaction mixture by filtration. Compound 5 ML L-DTTA salt may be isolated from the mother liquor and may be recycled by a process as described below. In particular, Compound 5 ML DTTA salt may be converted to racemic Compound 4 by a process comprising:
(i) converting Compound 5 ML L-DTTA salt to Compound 5 ML;
(ii) chlorination of Compound 5 ML to form ethyl 2-(4-((tert-butoxycarbonyl)amino)phenyl)-1-chloropiperidine-3-carboxylate (Compound 4-Cl):

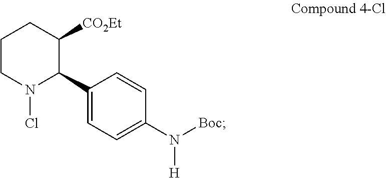 [SMILES: CCOC(=O)[C@@H]1CCCN(Cl)[C@@H]1c1ccc(NC(=O)OC(C)(C)C)cc1]

(iii) dechlorination (i.e. dehydrohalogenation) of Compound 4-Cl to form Compound 5A:

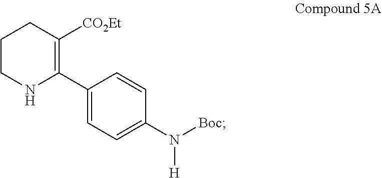 [SMILES: CCOC(=O)C1=C(c2ccc(NC(=O)OC(C)(C)C)cc2)NCCC1]

and
(iv) hydrogenation of Compound 5A to form Compound 4. The resulting Compound 4 may be subjected to the above steps (a)-(c) thereby increasing the yield of the desired isomer Compound 5.

The present disclosure further encompasses a process for preparing Avacopan, particularly to an optical resolution of racemic ethyl 2-(4-((tert-butoxycarbonyl)amino)phenyl)piperidine-3-carboxylate, an intermediate in the synthesis of Avacopan, into its desired isomer, ethyl (2R,3S)-2-(4-((tert-butoxycarbonyl)amino)-phenyl)piperidine-3-carboxylate.

In this process, the undesired isomer, ethyl (2S,3R)-2-(4-((tert-butoxycarbonyl)amino)phenyl)piperidine-3-carboxylate, is further recycled from the mother liquor.

The present disclosure encompasses process for preparing Avacopan. Particularly, it provides a process for increasing the enantiomeric purity of Avacopan intermediate, Compound 5, in a high yield.

The process for preparing Avacopan may comprise:
(a-i) preparing a mixture comprising Compound 5 as L-DTTA salt (Compound 5 L-DTTA) and Compound 5-ML as L-DTTA salt (Compound 5-ML L-DTTA);
(b-i) optionally basifying the L-DTTA salt of compound 5 followed by isolation of Compound 5, and subsequently reacting it in next synthetic step;
(c-i) basifying Compound 5-ML L-DTTA salt to obtain Compound 5-ML;
(d-i) chlorinating Compound 5-ML to obtain Compound 4-Cl;
(e-i) converting Compound 4-Cl to Compound 5A;
(f-i) converting Compound 5A to Compound 4;
(g-i) reacting compound 4 with L-DTTA to prepare a mixture comprising Compound 5 as L-DTTA salt and Compound 5-ML as L-DTTA salt; as in step a)
(h-i) optionally repeating steps (b) to (g); and
(i-i) converting Compound 5 obtained in each cycle, to Avacopan.

Advantageously, the process may be continuous, i.e. optical resolution of Compound 4 to obtain a mixture of Compound 5/Compound 5-ML as the L-DTTA salts, (wherein preferably compound 5 L-DTTA is precipitated and isolated from the reaction as a solid, and wherein preferably compound 5 ML L-DTTA is in the mother liquor), wherein Compound 5 is converted to Avacopan as described herein, whilst Compound 5 ML is recycled by converting back to Compound 4 for one or more cycles of optical resolution, recovery of Compound 5 and converting to Avacopan.

In the process of the present disclosure, Compound 4 is first reacted with L-Tolyl tartaric acid (“L-DTTA”) to form the L-DTTA salt of the desired (2S, 3R) isomer, i.e. Compound 5. The salt can be basified to obtain Compound 5, which is further reacted to obtain Avacopan. The second isomer, Compound 5-ML, is further recycled from the mother liquor.

The recycling of Compound 5-ML is performed by chlorinating this compound the obtain ethyl 2-(4-((tert-butoxycarbonyl)amino)phenyl)-1-chloropiperidine-3-carboxylate, Compound-4 Cl, followed by dechlorination (dehydrochlorination) to obtain a tetrahydropyridine compound, Compound 5A. Compound 5A is further transformed to Compound 4, which can again be resolved until a desired enantiomeric purity and yield are achieved.

The chlorination and dechlorination steps are illustrated by the following Scheme 3:

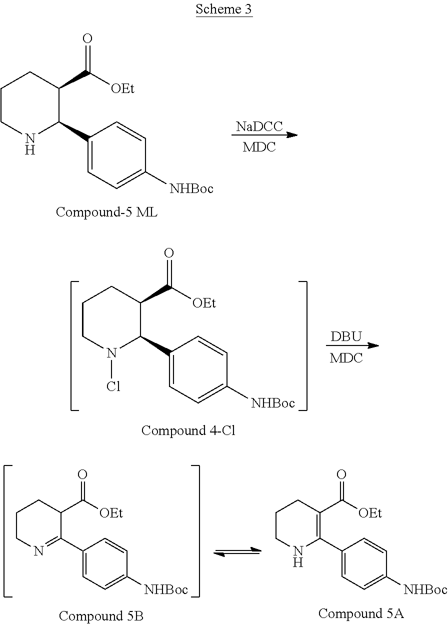

In certain embodiments, the present disclosure encompasses Compound 4-Cl. The compound can be isolated, or alternatively, can be used directly in the herein described process.

The present disclosure encompasses Compound 4-Cl for use in a process for preparing Avacopan. The process can be performed as described in the present disclosure or by other suitable processes.

The chlorination is typically performed using a chlorination agent, such as sodium hypochlorite (“NaOCl”), sodium dichloroisocyanurate (“NaDCC”), NaOCl/Tempo or N-Chloro succinamide (“NCS”); preferably it is NaOCl or NaDCC. The reaction can be done in the presence of a solvent, for example methylene dichloride (“DCM”) or toluene.

The obtained Compound 4-Cl is then converted to a teterahydropyridine compound—Compound 5A. In certain embodiments, compound 5A may exists in equilibrium as either enamine compound, Compound 5A and as imine compound, Compound 5B; preferably, it exist mostly as Compound 5A.

The conversion of Compound 4-Cl to Compound 5A is performed via a catalyst, for example 1,8-Diazabicyclo[5.4.0]undec-7-ene (“DBU”). It is usually done in a solvent, for example the solvent used in the previous step—like DCM or toluene.

Compound 5A is then converted back to Compound 4. This conversion is done using a catalyst, for example Pd/C, Pt₂O or Raney nickel in acetic acid.

The above described process can be illustrated by the following Scheme 4:

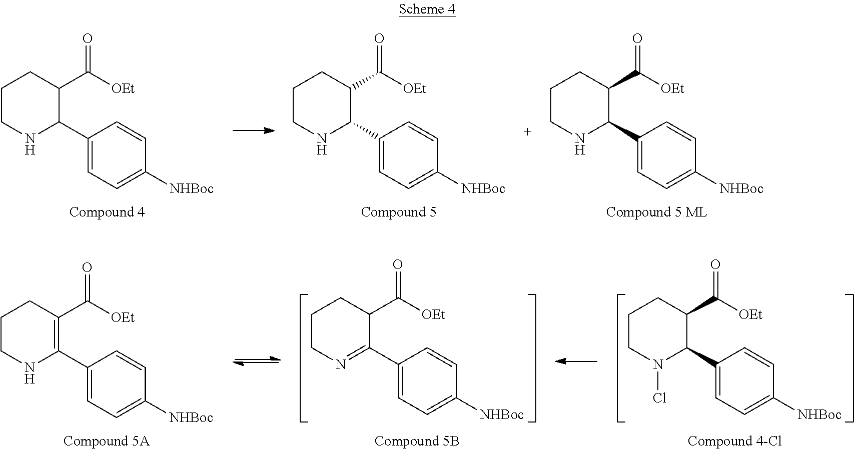

When the desired purity and yield are achieved, Compound 5 is used further to prepare Avacopan. The preparation of Avacopan can be done by any method disclosed in the literature, for example as described in WO 2010/075257.

In one embodiment, the present disclosure encompasses process for preparing Avacopan, comprising recycling of ethyl (2S,3R)-2-(4-((tert-butoxycarbonyl)amino)phenyl)piperidine-3-carboxylate (“Compound 5-ML”).

In certain embodiment, the present disclosure encompasses a process for preparing Avacopan, comprising:
a. Preparing a mixture comprising Compound 5 as L-DTTA salt and Compound 5-ML as DTTA salt;
b. Optionally basifying the L-DTTA salt of compound 5 followed by isolation of Compound 5, and subsequently reacting it in next synthetic step;
c. Basifying Compound 5-ML DTTA salt to obtain Compound 5-ML;
d. Chlorinating Compound 5-ML to obtain Compound 4-Cl;
e. Converting Compound 4-Cl to Compound 5A;
f. Converting Compound 5A to Compound 4;
g. Reacting compound 4 with L-DTTA to prepare a mixture comprising Compound 5 as L-DTTA salt and Compound 5-ML as DTTA salt; as in step a)
h. Optionally repeating steps b. to g.; and
i. Converting Compound 5 obtained in each cycle, to Avacopan.

It is understood that by referring to “cycle” it is meant the process starting from Compound 4 which is converted according to step a) to a mixture of Compound 5 and Compound 5-ML; followed by further reacting Compound 5 to obtain Avacopan according to step b) while recycling Compound 5-ML back to Compound 4, as described in the process steps c) to f) above.

The above described process is typically a continuous process, i.e. Compound 4 is prepared and further reacted to obtain Compound 5 and Compound 5-ML as their L-DTTA and DTTA salts, respectively, and the subsequent synthetic steps for preparing Avacopan are performed simultaneously while recycling Compound 5-ML back to Compound 4, which is again reacted and recycled, for unlimited cycles. Preferably, repeating the recycle process, i.e. steps b. to f) can be done for unlimited number of times, until a desired yield is achieved.

As mentioned above, converting Compound 5 to Avacopan can be done by any method. For example, it can be done by a process comprising:
a. reacting Compound 5 with 2-fluoro-6-methylbenzoyl chloride to obtain Compound 7;
b. deprotecting Compound 7 to obtain Compound 8;
c. reacting Compound 8 with Cyclopentanone to obtain Compound 10; and
d. Converting Compound 10 to Avacopan

The conversion of Compound 10 to Avacopan may comprise hydrolysis of Compound 10 to Compound 10-OH, and subsequently converting Compound 10-OH to Avacopan.

The above conversion of Compound 5 to Avacopan can be illustrated by the following Scheme 5:

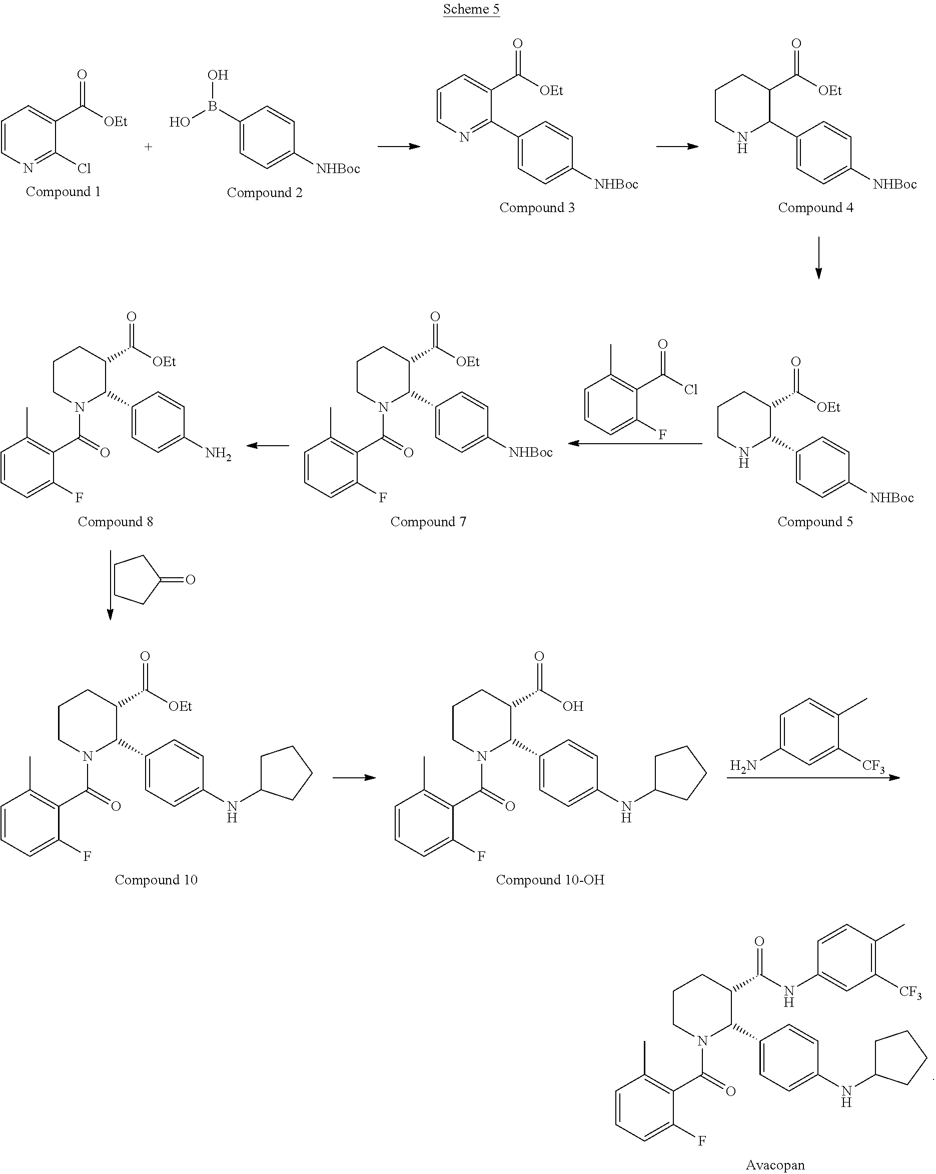

In the above process, preferred conditions can be utilized to afford efficient, short and ecological process.

Preferably, preparation of Compound 7 as specified in step a) is done using Toluene-water, in the presence of sodium carbonate. Typical reaction time can be from about 0.5 hour to about 3 hours, preferably 1 hour.

In the following step b), Compound 8 is typically prepared by deprotection of Compound 7, this can be done using aqueous hydrochloride and toluene as a solvent. Following this step, the obtained compound 8 is converted to Compound 10, according to step c), by reacting Compound 8 and cyclopentanone. Preferably, this reaction is performed using toluene as a solvent. In order to avoid formation of process impurities of Compound 8, Hantzsch ester (i.e. 1,4-dihydro-2,6-dimethyl-3,5-pyridinedicarboxylic acid diethyl ester) and p-toluene sulfonic acid (PTSA) can be added to Compound 10 using aqueous sulfuric acid to get Compound 10-OH. Compound 10-OH is converted to Avacopan by reacting with 4-Methyl-3-(trifluoromethyl)aniline using toluene as solvent and sodium carbonate as base.

The conversation of Compound 10 to Avacopan can be done by reacting this compound with 4-methyl-3-(trifluoromethyl)aniline. Typically, this reaction is done in the presence of trimethyl aluminum.

Alternatively, the present disclosure provides a process for preparing Avacopan comprising converting Compound 10 to Compound 10-OH, followed by reaction with 4-methyl-3-(trifluoromethyl)aniline to obtain Avacopan. Advantageously, this process avoids the use of hazardous reagents, such as trimethyl aluminum.

The present disclosure also provides another alternative for the conversion of Compound 10 to Avacopan. In this process, Compound 10 is converted to Compound 10-OH, followed by forming a Cl-intermediate, Compound 10-Cl, which is converted to Avacopan, for example by reacting Compound 10-Cl with 4-methyl-3-(trifluoromethyl)aniline.

Typically, Compound 10-OH is converted to Compound 10-Cl by reacting it with chlorinating agent, such as thionyl chloride or oxalyl chloride. Compound 10-Cl may be reacted directly to form Avacopan (“one pot”). In other embodiment, Compound 10-Cl may first be isolated, prior to converting it to Avacopan.

Alternatively, converting Compound 5 to Avacopan can be done by a process comprising converting Compound 7 into Compound 8-OH, which is then converted directly to compound 10-OH and then Avacopan. The process comprises:
a. reacting Compound 5 with 2-fluoro-6-methylbenzoyl chloride to obtain Compound 7;
b. Converting Compound 7 to obtain Compound 8-OH;
c. reacting Compound 8-OH with cyclopentanone to obtain Compound 10-OH; and
d. reacting Compound 10-OH with 4-methyl-3-(trifluoromethyl)aniline to obtain Avacopan.

This alternative conversion of Compound 5 to Avacopan can be illustrated by the following Scheme 6:

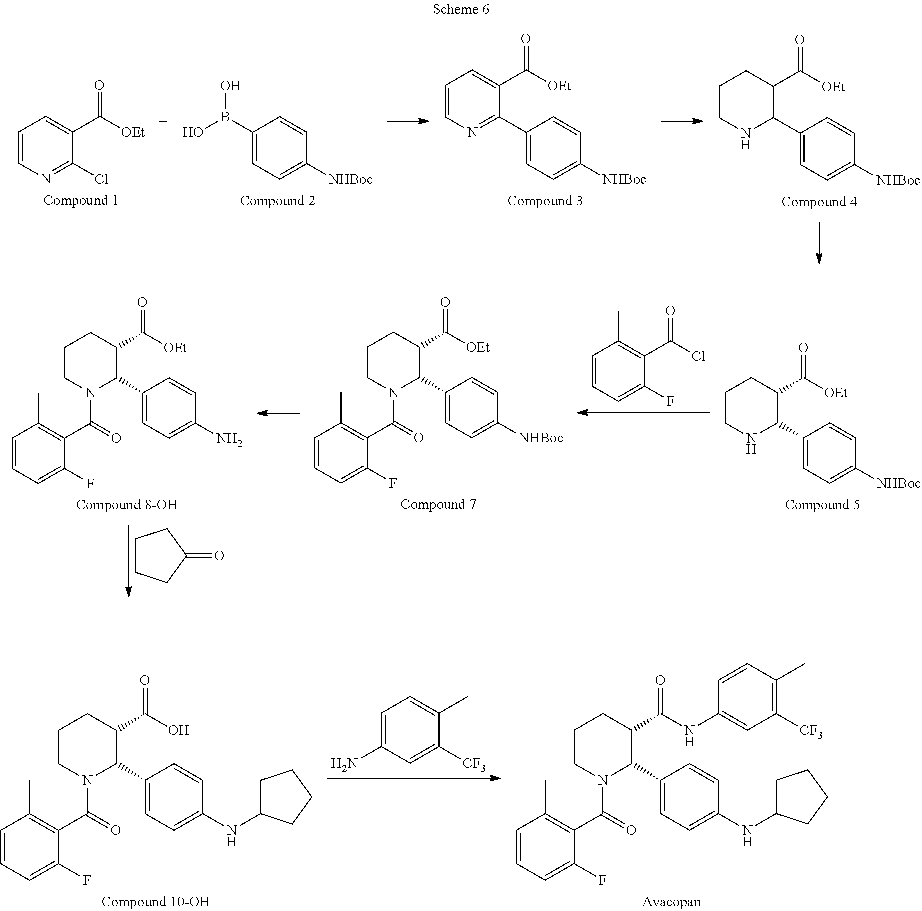

In the above described process, Compound 4 can be prepared as described in the literature, for example by reacting Compound 3 and a catalyst. In the present disclosure, the catalyst is preferably with Platinum oxide or Pd/C or Raney Nickel.

In a second aspect, the present disclosure provides a process for preparing Avacopan which also avoids the use of trimethyl aluminum. This process is done using the intermediate Compound 32. In this process, the coupling of the trifluoromethyl aniline is done on an early intermediate, followed by converting the pyridine moiety to piperidine and then optical resolution with L-DTTA. Later, the isomer is reacted with 2-fluoro-6-methylbenzoyl chloride, followed by deprotection to obtain Compound 32 and lastly coupling with cyclopentanone to obtain Avacopan.

The process can be illustrated by the following Scheme 7:

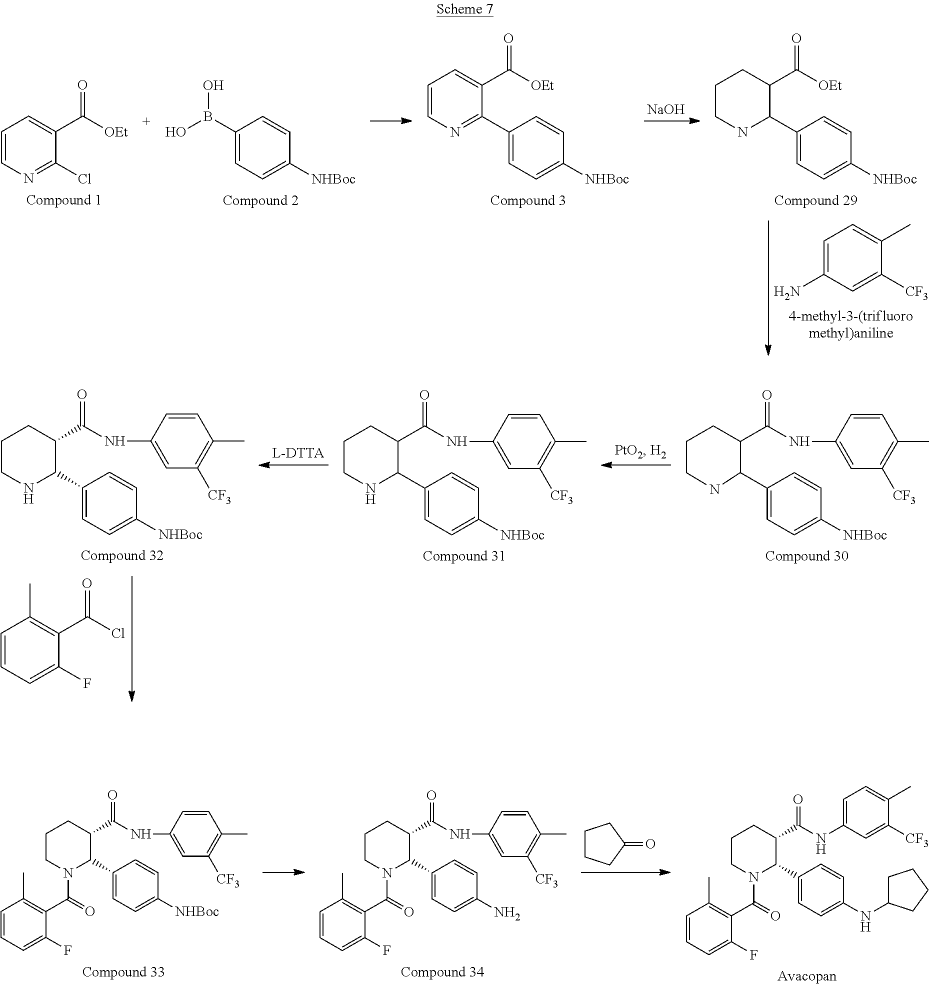

Alternatively, Compound 32 can be prepared by first converting of the pyridine moiety to piperidine and then coupling the aniline followed by optical resolution. The obtained Compound 32 is then converted to Avacopan.

The process can be illustrated by the following Scheme 8:

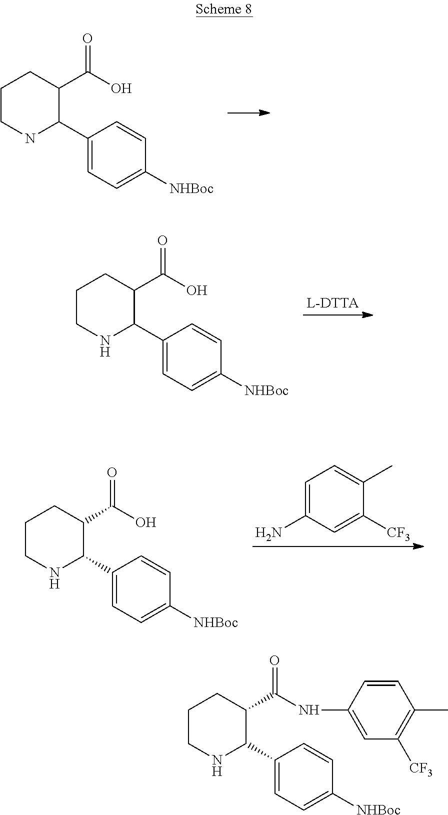

Avacopan prepared by all of the above described process, may be further purified. The present disclosure provides a simple, efficient and ecological purification process comprising crystallization from methanol and water solvent system. Preferably, the volume ratio of methanol to water is from about 10:1 to 5:1.

In specific embodiment, the present disclosure provides the following Avacopan dimer impurity, as well as composition comprising Avacopan and Avacopan dimer impurity in amount of not more than about 0.15% (w/w).

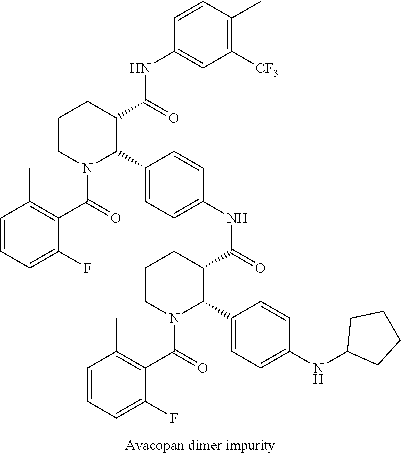

Avacopan dimer impurity may be isolated, it can also be in a crystalline form. Avacopan dimer impurity may be characterized by the following:

MS (ESI+) C₅₃H₅₄F₅N₅O₄ requires 919. Found: 920 [M+H]⁺.

¹H-NMR (400 MHz, DMSO-d₆) δ ppm 10.44 (s, 1H), 10.14 (m, 1H), 7.91 (m, 1H), 7.70 (m, 1H), 7.45-6.31 (m, 17H), 5.49 (m, 1H), 3.18-2.92 (brm, 6H), 2.34 (s, 6H), 2.18-1.38 (brm, 21H).

In specific embodiment, the present disclosure provides the following Avacopan sulfonamide impurity, as well as composition comprising Avacopan and Avacopan sulfonamide impurity in amount of not more than about 0.15% (w/w).

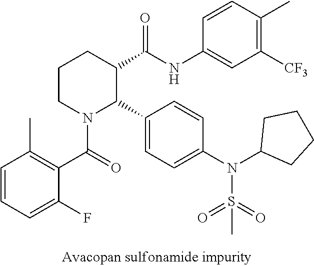

Avacopan sulfonamide impurity may be isolated, it can also be in a crystalline form. Avacopan sulfonamide impurity may be characterized by the following:

MS (ESI+) C₃₄H₃₇F₄N₃O₄S requires 659. Found: 660 [M+H]⁺.

¹H-NMR (400 MHz, DMSO-d₆) δ ppm 10.47 (s, 1H), 7.88 (q, 1H), 7.67 (dd, 1H), 7.48 (d, 2H), 7.33 (m, 1H), 7.31 (d, 1H), 7.24 (d, 2H), 7.17 (m, 1H), 7.08 (m, 1H), 6.49 (d, 1H), 3.23 (m, 2H), 3.23 (m, 1H), 3.03 (s, 3H), 2.37 (s, 3H), 2.35 (brm, 3H), 2.18 (m, 2H), 1.82, 1.42 (m, 4H), 1.42, 2.28 (m, 4H),

The present disclosure further provides Compound 4-Cl:

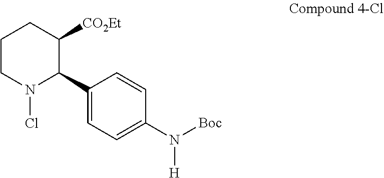 [SMILES: CCOC(=O)[C@@H]1CCCN(Cl)[C@@H]1c1ccc(NC(=O)OC(C)(C)C)cc1]

preferably in isolated form, preferably as a solid.

The present disclosure further provides Compound 5A:

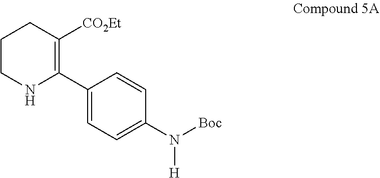 [SMILES: CCOC(=O)C1=C(c2ccc(NC(=O)OC(C)(C)C)cc2)NCCC1]

preferably in isolated form, preferably as a solid.

The present disclosure further provides the use of Compound 4-Cl of Compound 5A for the preparation of Avacopan.

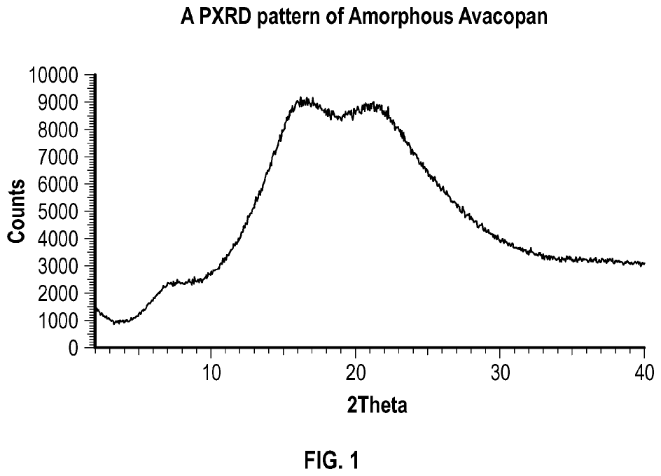

The present disclosure additionally provides amorphous Avacopan. Amorphous Avacopan may be characterized by having a powder X-ray diffractogram which is substantially free of distinct peaks. For example, amorphous Avacopan may be characterized by having broad peaks in the range of about 11 to about 30 degrees 2-theta±0.2 degrees 2-theta, optionally broad overlapping peaks at about 14 to 24 degrees 2-theta±0.2 degrees 2-theta, or an XRPD substantially as depicted in FIG. 1. Amorphous Avacopan may be prepared by precipitation from a mixture of water and methanol. Preferably, the water and methanol are in a ratio (v/v) of: about 1:2 to about 6:1, about 1:2 to about 5:1, about 1:1 to about 4:1, about 2:1 to about 4:1 or about 2.5:1 to about 3.5:1, or about 3:1. Preferably, amorphous Avacopan may be prepared by dissolving Avacopan in methanol and combining the solution with water. Preferably the solution of Avacopan in methanol is heated to a temperature of: about 30° C. to about 80° C., about 40° C. to about 70° C., about 45° C. to about 55° C. The solution may be optionally filtered to remove any insoluble particles. Preferably the solution is combined with water. The water may be at a temperature of: about −10° C. to about 30° C., about −8° C. to about 20° C., about −5° C. to about 15° C., about 0° C. to about 5° C. Preferably, the methanol solution is added to the water, wherein the water is cooled to about 0° C. to about 5° C. The mixture may be maintained for a suitable period of time, preferably for: about 30 minutes to 4 hours, about 1 hour to about 3 hours, about 1.5 hours to about 2.5 hours, or about 2 hours. The amorphous Avacopan may be isolated by filtration, and optionally dried.

Amorphous Avacopan may be used to prepare a pharmaceutical composition. The present disclosure encompasses a pharmaceutical composition comprising amorphous Avacopan as described herein and at least one pharmaceutically acceptable excipient. The amorphous Avacopan or pharmaceutical compositions thereof may be used as a medicament, particularly for the treatment of ANCA-associated Vasculitis. The present disclosure further provides a method of treatment of ANCA-associated Vasculitis, comprising administering a therapeutically effective amount of amorphous Avacopan of the present disclosure or a pharmaceutical composition thereof, to a subject in need of treatment.

Further aspects and embodiments of the present disclosure are set out in the following numbered clauses 1A-58A and 1B-85B.

### CLAUSES

1A. A process for kinetic resolution of a racemic amine, which is a racemic 2,3-disubstituted piperidine, by enzymatic oxidation to an imine or to an enamine or their mixture, comprising reacting the racemic 2,3-disubstituted piperidine with an imine reductase enzyme to form a mixture comprising enriched enantiomer of the amine, an imine and/or enamine.
2A. A process according to clause 1A wherein the kinetic resolution comprises oxidation of one isomer of the amine to form an imine and/or enamine and wherein one isomer of the amine is enriched.
3A. A process according to any of clauses 1A or 2A, wherein the kinetic resolution comprises:
(a) reacting a racemic amine having the formula:

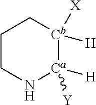

wherein X is alkyl, aryl or functional group, preferably wherein the functional group is an electron withdrawing group, and Y is H, C₁ to C₈ alkyl group, or aryl group, preferably a substituted phenyl, optionally wherein the substituent is selected from 4-amino, 4-t-butoxycarbonylamino, and 4-cyclopentylamino;
wherein one or both of C^{a} and C^{b} is a chiral center, to form a mixture comprising the chiral 2,3-disubstituted piperidine, imine, and enamine:

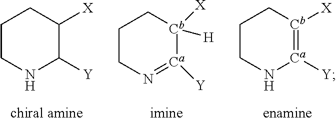

(b) optionally isolating the chiral amine; and
(c) optionally isolating the imine and/or enamine and converting the imine or enamine to the racemic amine.
   4A. A process according to clause 3A, wherein the chiral amine is isolated.
   5A. A process according to clause 3A or clause 4A, wherein X and Y are in a cis configuration.
   6A. A process according to any preceding clause wherein the racemic amine has the formula:

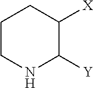

wherein
   X is C₁ to C₈ alkyl, C₆ to C₁₀ aryl or functional group, preferably an electron withdrawing group, particularly (C₁ to C₁₀ alkoxy)carbonyl, carboxyl, carbamoyl, cyano, formyl, C₁ to C₈ acyl, most preferably (C₁ to C₁₀ alkoxy)carbonyl;
   Y is H, C₁ to C₈ alkyl, or an optionally substituted C₆ to C₁₀ aryl, preferably a substituted phenyl, optionally wherein the substituent is selected from 4-amino, 4-t-butoxycarbonylamino, and 4-cyclopentylamino.
      7A. A process according to any preceding clause wherein the racemic amine has the formula:

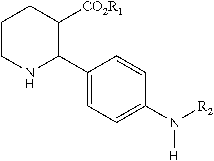

wherein
   R₁ is a C₁ to C₈ alkyl group, a or a C₁ to C₆ alkyl group; and
   R₂ is H, C₁ to C₆ alkyl group a C₅ to C₈ cycloalkyl group, or a protecting group, particularly wherein R₂ is cyclopentyl or t-butyloxycarbonyl (Boc), and more particularly Boc.
      8A. A process according to clause 7A wherein R₁ is a C₁ to C₃ alkyl group, and preferably wherein R₁ is ethyl, more preferably wherein R₁ is ethyl and R₂ is Boc.
      9A. A process according to any preceding clause wherein the reaction is carried out in the presence of a cofactor regenerating system containing NADPH, NADP+ or their mixture as cofactor, optionally wherein the cofactor regenerating system comprises: cofactor regenerating enzyme, such as an NADPH oxidase (NOX), an alcohol dehydrogenase (ADH) or a ketoreductase (KRED), preferably wherein the cofactor regenerating system comprises ADH or KRED wherein acetone is used as sacrificial substrate, most preferably comprises ADH from Lactobacillus brevis.
      10A. A process according to any preceding clause, wherein the imine reductase enzyme is related to SFam1 and related to Variovorax sp. (such as Variovorax beijingensis—SEQID2).
      11A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to SEQ ID 1:

(PRO-IRED-128)
SEQ ID 1
MTDVSLIGLGPMGMALARALQSSKFTLTVWNRTAERAKPVLNPGTVLAPT
ALAAVQASPVVLVCVADYPASRAILTAPGVHDALRGKVLVQLSTGTPQDA
RDDWAALSGVAYLDGALLATPGQIGRPDTPLFISGEARALAACRPLLEAI
AGNIQHMGEPIGNAAAWDLATLSCMFGAMSGFFHGVRICESEGLGVDAFS
QMIGAISPVLGEMISAEGEAIHANRYGEPESSMATCAGSGRLFVKQAREA
KLDASFPDFLMGLFERSLSAGFANERLAAMVKVMR

12A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to residues 1 to 60 of SEQ ID 1.
13A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to residues 61 to 120 of SEQ ID 1.
14A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to residues 121 to 180 of SEQ ID 1.
15A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to residues 181 to 240 of SEQ ID 1.
16A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to residues 241 to 285 of SEQ ID 1.
17A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to at least 50 contiguous amino acid residues of SEQ ID 1.
18A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to at least 100 contiguous amino acid residues of SEQ ID 1.
19A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to at least 150 contiguous amino acid residues of SEQ ID 1.
20A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to at least 200 contiguous amino acid residues of SEQ ID 1.
21A. A process according to any preceding clause, wherein the imine reductase enzyme has an amino acid sequence that comprises at least 80%, at least 85%, at least 90%, at least 92%, at least 95%, at least 96%, at least 97%, at least 98%, and at least 99% identity to at least 250 contiguous amino acid residues of SEQ ID 1.
22A. A process according to any preceding clause, wherein the imine reductase enzyme has an amino acid sequence according to SEQ ID 1 which comprises: one or more, five or more, ten or more, 15 or more, 20 or more, 25 or more, 30 or more, 35 or more, or 40, mutations at a position selected from 14, 21, 22, 25, 35, 38, 40, 43, 50, 69, 71, 73, 76, 81, 102, 108, 122, 137, 146, 150, 156, 191, 195, 197, 198, 200, 215, 224, 225, 251, 255, 262, 263, 265, 267, 269, 272, 273, 281, and 284.
23A. A process according to any of preceding clause, wherein the imine reductase enzyme has an amino acid sequence corresponding to SEQ ID 1, SEQ ID 2, SEQ ID 3, or SEQ ID 4.
24A. A process according to any preceding clause, further comprising ex situ recycling of the imine/enamine back to the racemic amine, preferably wherein the imine and/or enamine is isolated and converted to the racemic amine.
25A. A process according to clause 24A, wherein the imine and/or enamine is isolated by extraction, optionally in the presence of an acid or base, by precipitation and filtering, or by filtering from the reaction mixture, and optionally crystallising.
26A. A process according to any of clauses 24A or 25A, wherein the conversion of the imine and/or enamine to the racemic amine is carried out by catalytic hydrogenation, preferably wherein the catalyst is palladium on carbon, platinum (II) oxide, or wherein the catalyst Raney nickel and an organic acid.
27A. A process according to any of clauses 1A to 23A, further comprising dynamic kinetic enzymatic reduction of the imine and/or enamine to form the chiral amine, wherein the dynamic kinetic enzymatic reduction is carried out using an imine reductase enzyme.
28A. A process according to clause 27A, wherein the reaction is carried out in the presence of a cofactor regenerating system, containing NADPH, NADP+ or their mixture as cofactor, optionally wherein the cofactor regenerating enzyme is glucose dehydrogenase (GDH).
29A. A process according to clause 27A or clause 28A, wherein the imine reductase enzyme is related to SFam1 and Streptomyces sp. (such Streptomyces albus—SEQ ID 5; and Streptomyces rimosus SEQ ID 6).
30A. A process according to any of clauses 27A to 29A, wherein the enzyme has an amino acid sequence that comprises at least 80%, at least 85%, at least 90%, at least 92%, at least 95%, at least 96%, at least 97%, at least 98%, and at least 99% identity to SEQ ID 5.
31A. A process according to any of clauses 27A to 30A, wherein the imine reductase enzyme has an amino acid sequence that comprises at least 80%, at least 85%, at least 90%, at least 92%, at least 95%, at least 96%, at least 97%, at least 98%, and at least 99% identity to residues 1 to 60 of SEQ ID 5.
32A. A process according to any of clauses 27A to 31A, wherein the imine reductase enzyme has an amino acid sequence that comprises at least 80%, at least 85%, at least 90%, at least 92%, at least 95%, at least 96%, at least 97%, at least 98%, and at least 99% identity to residues 61 to 120 of SEQ ID 5.
33A. A process according to any of clauses 27A to 32A, wherein the imine reductase enzyme has an amino acid sequence that comprises at least 80%, at least 85%, at least 90%, at least 92%, at least 95%, at least 96%, at least 97%, at least 98%, and at least 99% identity to residues 121 to 180 of SEQ ID 5.
34A. A process according to any of clauses 27A to 33A, wherein the imine reductase enzyme has an amino acid sequence that comprises at least 80%, at least 85%, at least 90%, at least 92%, at least 95%, at least 96%, at least 97%, at least 98%, and at least 99% identity to residues 181 to 240 of SEQ ID 5.
35A. A process according to any of clauses 27A to 34A, wherein the imine reductase enzyme has an amino acid sequence that comprises at least 80%, at least 85%, at least 90%, at least 92%, at least 95%, at least 96%, at least 97%, at least 98%, and at least 99% identity to residues 241 to 295 of SEQ ID 5.
36A. A process according to any of clauses 27A to 35A, wherein the imine reductase enzyme has an amino acid sequence that comprises at least 80%, at least 85%, at least 90%, at least 92%, at least 95%, at least 96%, at least 97%, at least 98%, and at least 99% identity to at least 50 contiguous amino acid residues of SEQ ID 5.
37A. A process according to any of clauses 27A to 36A, wherein the imine reductase enzyme has an amino acid sequence that comprises at least 80%, at least 85%, at least 90%, at least 92%, at least 95%, at least 96%, at least 97%, at least 98%, and at least 99% identity to at least 100 contiguous amino acid residues of SEQ ID 5.
38A. A process according to any of clauses 27A to 37A, wherein the imine reductase enzyme has an amino acid sequence that comprises at least 80%, at least 85%, at least 90%, at least 92%, at least 95%, at least 96%, at least 97%, at least 98%, and at least 99% identity to at least 150 contiguous amino acid residues of SEQ ID 5.
39A. A process according to any of clauses 27A to 38A, wherein the imine reductase enzyme has an amino acid sequence that comprises at least 80%, at least 85%, at least 90%, at least 92%, at least 95%, at least 96%, at least 97%, at least 98%, and at least 99% identity to at least 200 contiguous amino acid residues of SEQ ID 5.
40A. A process according to any of clauses 27A to 39A, wherein the imine reductase enzyme has an amino acid sequence that comprises at least 80%, at least 85%, at least 90%, at least 92%, at least 95%, at least 96%, at least 97%, at least 98%, and at least 99% identity to at least 250 contiguous amino acid residues of SEQ ID 5.
41A. A process according to any of clauses 27A to 40A, wherein the imine reductase enzyme has an amino acid sequence corresponding to: SEQ ID 5 or SEQ ID 6.
42A. Use of a process as defined in any of the preceding clauses in the synthesis of Avacopan, wherein the racemic amine has the formula:

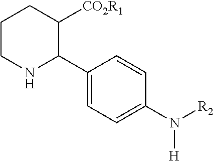

wherein
   R₁ is a C₁ to C₈ alkyl group, or a C₁ to C₆ alkyl group; and
   R₂ is H, a C₅ to C₈ cycloalkyl group, or a protecting group, particularly wherein R₂ is cyclopentyl or t-butyloxycarbonyl (Boc), and more particularly Boc.
      43A. Use according to clause 42A, wherein the racemic amine is compound 4 having the formula:

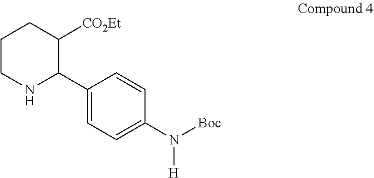 [SMILES: CCOC(=O)C1CCCNC1c1ccc(NC(=O)OC(C)(C)C)cc1]

44A. Use according to clause 42A or clause 43A, wherein the racemic amine is reacted with an imine reductase enzyme as defined in any of clauses 10A to 23A, preferably clauses 17A to 23A, 22A-23A, and particularly wherein the imine reductase enzyme has an amino acid sequence corresponding to: SEQ ID 1, SEQ ID 2, SEQ ID3, or SEQ ID 4.
45A. Use according to any of clauses 42A to 44A, wherein the reaction is carried out in the presence of a cofactor regenerating system, optionally wherein the cofactor regenerating enzyme is an NADPH oxidase (NOX), an alcohol dehydrogenase (ADH) or a ketoreductase (KRED), preferably ADH or KRED.
46A. Use according to any of clauses 42A to 45A, wherein the resulting enamine Compound 5A having the formula:

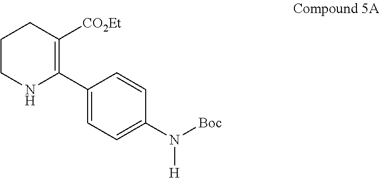 [SMILES: CCOC(=O)C1=C(c2ccc(NC(=O)OC(C)(C)C)cc2)NCCC1]

is isolated optionally by extraction, optionally in the presence of an acid or base, by precipitation and filtering, or by filtering from the reaction mixture, and optionally crystallising, and converted to the racemic amine.
   47A. Use according to clause 46A, wherein the conversion of the imine and/or enamine to the racemic amine is carried out by catalytic hydrogenation, preferably wherein the catalyst is palladium on carbon, platinum (II) oxide, or wherein the catalyst Raney nickel and an organic acid.
   48A. Use according to any of clauses 42A to 45A, wherein the imine and/or enamine is subjected to dynamic kinetic enzymatic reduction form the chiral amine, wherein the dynamic kinetic enzymatic reduction is carried out using an imine reductase enzyme, wherein the enzyme is as defined in any of clauses 29A to 41A, preferably clauses 31A to 41A, and more preferably clauses 36A-41A, and particularly wherein the imine reductase enzyme has an amino acid sequence corresponding to SEQ ID 5 or SEQ ID6.
   49A. A process according to any of clauses 42A to 48A, wherein the reaction is carried out in the presence of a cofactor regenerating system, optionally wherein the cofactor regenerating enzyme is glucose dehydrogenase (GDH).
   50A. A process for preparing Avacopan, comprising kinetic resolution of cis-ethyl 2-(4-((tert-butoxycarbonyl)amino)phenyl)piperidine-3-carboxylate (Compound 4):

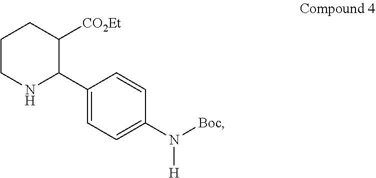 [SMILES: CCOC(=O)C1CCCNC1c1ccc(NC(=O)OC(C)(C)C)cc1]

to form a mixture comprising: ethyl (2R,3S)-2-(4-((tert-butoxycarbonyl)amino)phenyl)-piperidine-3-carboxylate (Compound 5) and ethyl 2-(4-((tert-butoxycarbonyl)amino)-phenyl)-1,4,5,6-tetrahydropyridine-3-carboxylate (Compound 5A):

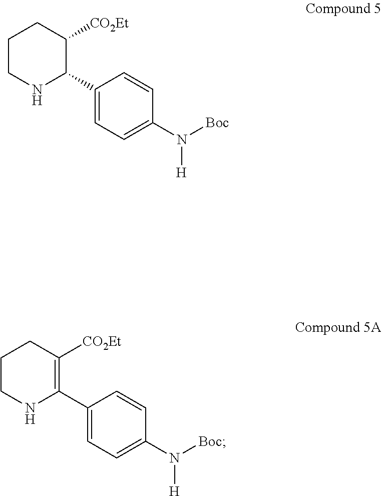 [SMILES: CCOC(=O)[C@H]1CCCN[C@H]1c1ccc(NC(=O)OC(C)(C)C)cc1 ; CCOC(=O)C1=C(c2ccc(NC(=O)OC(C)(C)C)cc2)NCCC1]

optionally using a cofactor regenerating enzyme, particularly selected from NADPH oxidase (NOX), alcohol dehydrogenase (ADH) or ketoreductase (KRED), preferably wherein the kinetic resolution of the cyclic amine compound is carried out by an imine reductase enzyme and ADH cofactor regenerating system.
   51A. A process according to clause 50A, wherein the cofactor regenerating enzyme is ADH or KRED and the kinetic resolution is carried out using a catalytic amount of nicotinamide adenine dinucleotide phosphate (NADP+) cofactor and acetone as sacrificial substrate.
   52A. A process according to clause 50A or clause 51A, comprising recycling Compound 5A via extraction at acidic pH or by precipitation and filtering, or by filtering from the reaction mixture, and optionally crystallising.
   53A. A process according to clause 50A or clause 51A, wherein Compound 5A is isolated by acid-base extraction, crystallized and filtered, or filtered from reaction mixture, thereby providing a solution comprising Compound 5, that can be converted to Avacopan, and optionally recycling the filtered Compound 5A to Compound 4.
   54A. A process according to any of clauses 50A to 53A, comprising crystallizing Compound 5A from the reaction mixture comprising Compound 5 and Compound 5A, isolating the crystallized Compound 5A and further converting it to Compound 4, and converting Compound 5 to prepare Avacopan.
   55A. A process according to any of clauses 50A to 54A, wherein Compound 5A is converted back to Compound 4, preferably by hydrogenation using a catalyst, and more preferably Pd/C, Pt₂O or Raney nickel in acetic acid.
   56A. A process according to any of clauses 50A to 54A, wherein Compound 5A is converted to Compound 5 by dynamic kinetic reduction, preferably using an imine reductase enzyme and glucose dehydrogenase (GDH) cofactor regenerating enzyme.
   57A. A process according to any of clauses 50A to 56A wherein the kinetic resolution of the cyclic amine is carried out using an imine reductase enzyme as defined in any of clauses 10A to 23A.
   58A. A process according to clause 56A, wherein the Compound 5A is converted to Compound 5 by dynamic kinetic enzymatic reduction using an imine reductase enzyme as defined in any of clauses 29A to 41A.
   1B. A process for the optical resolution of racemic ethyl 2-(4-((tert-butoxycarbonyl)-amino)phenyl)piperidine-3-carboxylate (Compound 4):

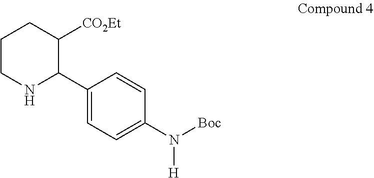 [SMILES: CCOC(=O)C1CCCNC1c1ccc(NC(=O)OC(C)(C)C)cc1]

comprising:
   (a) reacting Compound 4 with L-tolyl tartaric acid (L-DTTA);
   (b) separating the L-DTTA salt of ethyl (2R,3 S)-2-(4-((tert-butoxycarbonyl)amino)phenyl)piperidine-3-carboxylate (Compound 5 L-DTTA salt) from the L-DTTA salt of ethyl (2S,3R)-2-(4-((tert-butoxycarbonyl)amino)phenyl)piperidine-3-carboxylate (Compound 5-ML L-DTTA salt):

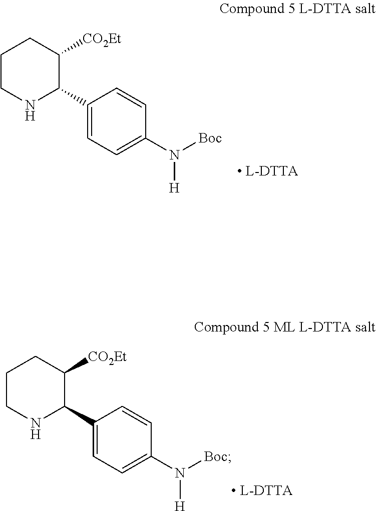 [SMILES: CCOC(=O)[C@H]1CCCN[C@H]1c1ccc(NC(=O)OC(C)(C)C)cc1 ; CCOC(=O)[C@@H]1CCCN[C@@H]1c1ccc(NC(=O)OC(C)(C)C)cc1]

(c) recycling Compound 5 ML L-DTTA salt by converting to racemic Compound 4.
   2B. A process according to clause 1B, wherein step (b) comprises isolating Compound 5 L-DTTA salt from the reaction mixture, preferably by filtration.
   3B. A process according to clause 1B or clause 2B, wherein the reaction in step (a) is carried out in a solvent, preferably wherein the solvent is an alcohol, more preferably a C₁ to C₆ alcohol, more preferably a C₁ to C₃ alcohol, and particularly ethanol.
   4B. A process according to any of clauses 1B to 3B, wherein the reaction in step (a) is carried out at a temperature of: about 30° C. to about 100° C., about 40° C. to about 80° C., about 45° C. to about 60° C., or about 50° C. to about 55° C.
   5B. A process according to any of clauses 1B to 4B, wherein Compound 5 L-DTTA salt is precipitated from a solvent mixture comprising an ester and an ether, preferably wherein the ester is a C₄ to C₈ ester, more preferably a C₄ to C₆ ester, and particularly isopropyl acetate; and/or wherein the ether is a preferably wherein the ester is a C₄ to C₈ ester and particularly methyl tert-butyl ether (MTBE); and most preferably wherein Compound 5 L-DTTA salt is precipitated from a mixture of isopropyl acetate and MTBE.
   6B. A process according to any of clauses 1B to 5B, wherein the Compound 5 L-DTTA salt is recrystallized from a solvent mixture comprising an ester and an ether, preferably wherein the ester is a C₄ to C₈ ester, more preferably a C₄ to C₆ ester, and tetrahydrofuran (THF); and/or wherein the ether is a preferably wherein the ester is a C₄ to C₈ ester and particularly methyl tert-butylether (MTBE); and most preferably wherein Compound 5 L-DTTA salt is recrystallized from a mixture of THE and MTBE.
   7B. A process according to any of clauses 2B to 6B, wherein Compound 5 ML L-DTTA salt is isolated from the mother liquor. 8B. A process according to any of clauses 1B to 7B, wherein step (c) comprises:
(i) converting Compound 5 ML L-DTTA salt to Compound 5 ML;
(ii) chlorination of Compound 5 ML to form ethyl 2-(4-((tert-butoxycarbonyl)amino)phenyl)-1-chloropiperidine-3-carboxylate (Compound 4-Cl):

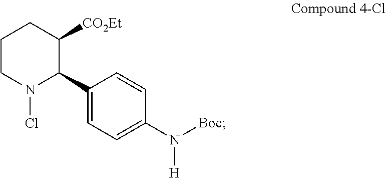 [SMILES: CCOC(=O)[C@@H]1CCCN(Cl)[C@@H]1c1ccc(NC(=O)OC(C)(C)C)cc1]

(iii) dechlorination (dehydrohalogenation) of Compound 4-Cl to form Compound 5A:

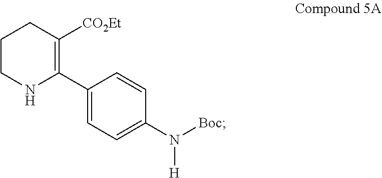 [SMILES: CCOC(=O)C1=C(c2ccc(NC(=O)OC(C)(C)C)cc2)NCCC1]

and
(iv) hydrogenation of Compound 5A.
   9B. A process according to any of clauses 1B to 8B, further comprising repeating steps (a)-(c) using the racemic Compound 4 from recycling step (c).
   10B. A process according to any of clauses 8B or 9B, wherein step (i) comprises reacting Compound 5 ML L-DTTA salt with a base, preferably an inorganic base, more preferably wherein the base is alkaline metal carbonate or alkaline metal hydrogencarbonate; and particularly sodium carbonate, potassium carbonate, sodium hydrogencarbonate or potassium hydrogen carbonate, more particularly sodium hydrogen carbonate.
   11B. A process according to clause 10B, wherein step (i) is carried out in a solvent, preferably selected from the group consisting of chlorinated hydrocarbons, esters, and ethers; preferably wherein the chlorinated hydrocarbon is a chlorinated C₁ to C₆ hydrocarbon or chlorinated C₁ to C₃ hydrocarbon, and particularly dichloromethane; or wherein the ester is a C₄ to C₈ ester, more preferably a C₄ to C₆ ester; or wherein the ether is a C₄ to C₈ ether and particularly methyl tert-butylether (MTBE); and most preferably wherein the solvent is dichloromethane.
   12B. A process according to any of clauses 8B to 11B, wherein step (ii) is carried out using a chlorination agent preferably selected from sodium hypochlorite, sodium dichloroisocyanurate, sodium hypochlorite/(2,2,6,6-Tetramethylpiperidin-1-yl)oxyl or (2,2,6,6-tetramethylpiperidin-1-yl)oxidanyl (TEMPO), and N-chlorosuccinimide, preferably sodium hypochlorite or sodium dichloroisocyanurate.
   13B. A process according to clause 12B, wherein the chlorinating agent is sodium hypochlorite or sodium dichloroisocyanurate.
   14B. A process according to clause 13B, wherein the chlorinating agent is sodium hypochlorite and the reaction is carried out in one or more solvents selected from the group consisting of a chlorinated hydrocarbon, an alcohol and water; preferably wherein the chlorinated hydrocarbon is a chlorinated C₁ to C₆ hydrocarbon or chlorinated C₁ to C₃ hydrocarbon, and particularly dichloromethane; and preferably wherein the alcohol is a C₁ to C₆ alcohol, more preferably a C₂ to C₄ alcohol, and particularly t-butanol; and most preferably wherein the reaction is carried out in a solvent mixture comprising dichloromethane, t-butanol and water.
   15B. A process according to clause 14B, wherein the reaction in step (ii) is carried out in the presence of an organic acid, preferably a C₂ to C₆ carboxylic acid, more preferably a C₃ to C₄ carboxylic acid, and particularly acetic acid.
   16B. A process according to any of clauses 14B or 15B, wherein the reaction in step (ii) is carried out at a temperature of about −10° C. to about 25° C., about −5° C. to about 20° C., about −5° C. to about 15° C. or about 0 to about 10° C.
   17B. A process according to clause 13B, wherein the chlorinating agent is sodium dichloroisocyanurate, and the reaction is carried out in one or more solvents selected from the group consisting of a hydrocarbon, and water; preferably wherein the hydrocarbon is a C₆ to C₁₀ preferably aromatic, hydrocarbon or a C₆ to C₈, preferably aromatic hydrocarbon, and particularly toluene; and most preferably wherein the reaction is carried out in a solvent mixture comprising toluene and water.
   18B. A process according to clause 17B, wherein the reaction in step (ii) is carried out at a temperature of about 10° C. to about 80° C., about 15° C. to about 60° C., about 15° C. to about 50° C., or about 20° C. to about 30° C.
   19B. A process according to any of clauses 8B to 18B, wherein the Compound 4-Cl is isolated as a solution from the reaction in step (ii) and is reacted in step (iii) as the solution.
   20B. A process according to any of clauses 8B to 19B, wherein step (iii) comprises reacting Compound 4-Cl with base, preferably a non-nucleophilic base, and more preferably wherein the is base selected from the group consisting of: 1,8-Diazabicyclo[5.4.0]undec-7-ene (“DBU”), sodium ethoxide, potassium tert-butoxide, butyl lithium, sodium amide, methyl lithium, and tert-butyllithium, particularly wherein the base is: 1,8-Diazabicyclo[5.4.0]undec-7-ene.
   21B. A process according to any of clauses 8B to 20B, wherein the reaction in step (iii) is carried out in a solvent, preferably wherein the solvent comprises methylene dichloride and/or toluene.
   22B. A process according to any of clauses 8B to 21B, wherein the reaction in step (iii) is carried out at a temperature of about 10° C. to about 60° C., about 15° C. to about 50° C., about 15° C. to about 40° C., or about 20° C. to about 30° C.
   23B. A process according to any of clauses 8B to 22B, wherein Compound 4-Cl is not isolated as a solid from the reaction solvent in step (ii).
   24B. A process according to any of clauses 8B to 23B, wherein Compound 4-Cl is extracted from the reaction mixture in step (ii) as a solution, and wherein step (iii) is carried out on the solution.
   25B. A process according to any of clauses 8B to 24B, wherein step (iv) comprises reacting Compound 5A with hydrogen in the presence of a hydrogenation catalyst, preferably wherein step (iv) comprise hydrogenation of Compound 5A in the presence of a catalyst selected from palladium on carbon, platinum (II) oxide, Raney nickel in acetic acid, and more preferably wherein the hydrogenation catalyst is palladium on carbon.
   26B. A process according to clause 25B, wherein the catalyst is palladium on carbon.
   27B. A process according to clause 25B or clause 26B, wherein step (iv) is carried out in a solvent which is preferably an alcohol, more preferably a C₁ to C₆ alcohol, most preferably a C₂ to C₄ alcohol, and particularly isopropanol.
   28B. A process according to any of clauses 25B to 27B, wherein step (iv) is carried out in the presence of an organic acid, preferably a C₂ to C₆ carboxylic acid, more preferably a C₃ to C₄ carboxylic acid, and particularly acetic acid.
   29B. A process according to any of clauses 25B to 28B, wherein the reaction in step (iv) is carried out at a temperature of: about 30° C. to about 90° C., about 40° C. to about 80° C., about 55° C. to about 75° C., or about 55° C. to about 65° C.
   30B. A process according to any preceding clause further comprising reacting Compound 5 L-DTTA salt with a base, preferably an inorganic base, more preferably wherein the base is alkaline metal carbonate or alkaline metal hydrogencarbonate; and particularly sodium carbonate, potassium carbonate, sodium hydrogencarbonate or potassium hydrogen carbonate, more particularly sodium hydrogen carbonate to form Compound 5.
   31B. A process according to clause 30B wherein the reaction is carried out in a solvent selected from an ether, a chlorinated hydrocarbon, an ester or a hydrocarbon; preferably wherein the ether is a C₄ to C₈ ether and particularly methyl tert-butylether (MTBE); or wherein the chlorinated hydrocarbon is a chlorinated C₁ to C₆ hydrocarbon or a chlorinated C₁ to C₃-hydrocarbon, and particularly dichloromethane; or wherein the ester is a C₄ to C₈ ester, more preferably a C₂ to C₆ ester and particularly isopropyl acetate or ethyl acetate; or wherein the hydrocarbon is a C₆ to C₁₀ preferably aromatic, hydrocarbon or a C₆ to C₈, preferably aromatic hydrocarbon, and particularly toluene.
   32B. A process according to clause 31B, wherein the reaction is carried out in dichloromethane.
   33B. A process for preparing Avacopan comprising optical resolution of racemic Compound 4 according to the process of any of clauses 1B to 32B.
   34B. A process according to clause 33B, wherein the Compound 4 recycled from step (c) is subjected to optical resolution comprising with L-tolyl tartaric acid (L-DTTA); separating Compound 5 L-DTTA salt from Compound 5-ML L-DTTA salt; and converting Compound 5-ML L-DTTA salt to Compound 4; and optionally repeating the cycle of optical resolution of Compound 4 and converting the Compound 5-ML L-DTTA salt to Compound 4.
   35B. A process according to any of clauses 1B to 34B, wherein the Compound 5 L-DTTA salt from the recycling of Compound 4 is converted to Avacopan.
   36B. A process according to any of clauses 33B to 35B, comprising:
(a-i) preparing a mixture comprising Compound 5 as L-DTTA salt and Compound 5-ML as DTTA salt;
(b-i) optionally basifying the L-DTTA salt of compound 5 followed by isolation of Compound 5, and subsequently reacting it in next synthetic step;
(c-i) basifying Compound 5-ML DTTA salt to obtain Compound 5-ML;
(d-i) chlorinating Compound 5-ML to obtain Compound 4-Cl;
(e-i) converting Compound 4-Cl to Compound 5A;
(f-i) converting Compound 5A to Compound 4;
(g-i) reacting compound 4 with L-DTTA to prepare a mixture comprising Compound 5 as L-DTTA salt and Compound 5-ML as DTTA salt; as in step a)
(h-i) optionally repeating steps (b) to (g); and
(i-i) converting Compound 5 obtained in each cycle, to Avacopan.
   37B. A process according to any of clauses 33B to 36B, which is a continuous process, wherein Compound 4 is prepared and subjected to optical resolution to obtain a mixture of Compound 5 and Compound 5-ML as their L-DTTA salts, wherein Compound 5 is converted to Avacopan and Compound 5-ML is recycled by converting back to Compound 4 for one or more cycles of optical resolution, recovery of Compound 5 from the recycling steps and converting to Avacopan.
   38B. A process according to clause 36B or clause 37B, wherein Compound 5 is converted to Avacopan by a process comprising:
(a-ii) reacting Compound 5 with 2-fluoro-6-methylbenzoyl chloride to obtain Compound 7

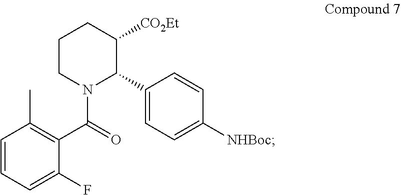 [SMILES: CCOC(=O)[C@H]1CCCN(C(=O)c2c(C)cccc2F)[C@H]1c1ccc(NC(=O)OC(C)(C)C)cc1]

(b-ii) deprotecting Compound 7 to obtain Compound 8:

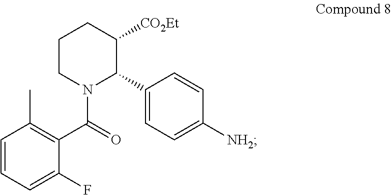 [SMILES: CCOC(=O)[C@H]1CCCN(C(=O)c2c(C)cccc2F)[C@H]1c1ccc(N)cc1]

(c-ii) reacting Compound 8 with cyclopentanone to obtain Compound 10:

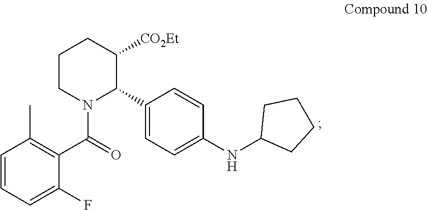 [SMILES: CCOC(=O)[C@H]1CCCN(C(=O)c2c(C)cccc2F)[C@H]1c1ccc(NC2CCCC2)cc1]

and
(d-ii) converting Compound 10 to Avacopan.
   39B. A process according to clause 38B, wherein step (a-ii) is carried out in a solvent comprising toluene and water, in the presence of a base, preferably an inorganic base, more preferably an alkali metal carbonate or an alkaline metal bicarbonate, and more preferably sodium carbonate or potassium carbonate, particularly sodium carbonate.
   40B. A process according to clause 38B or clause 39B, wherein step (b-ii) comprises reacting Compound 7 with a mineral acid, optionally an aqueous mineral acid, preferably wherein the acid is hydrochloric acid, and optionally in a solvent, preferably a hydrocarbon such as a C₆ to C₁₀ preferably aromatic, hydrocarbon or a C₆ to C₈, preferably aromatic hydrocarbon, and particularly toluene.
   41B. A process according to any of clauses 38B to 40B, wherein step (c-ii) comprises reacting Compound 8 with cyclopentanone using Hantzsch ester, preferably in the presence of an acid, more preferably wherein the acid is p-toluene sulfonic acid, acetic acid or methane sulfonic acid.
   42B. A process according to clause 41B, wherein the reaction is performed in a solvent, preferably a hydrocarbon such as a C₆ to C₁₀ preferably aromatic, hydrocarbon or a C₆ to C₈, preferably aromatic hydrocarbon, and particularly toluene.
   43B. A process according to any of clauses 38B to 42B, wherein step (d-ii) comprises hydrolysis of Compound 10 using a mineral acid, preferably sulfuric acid or hydrochloric acid, more preferably sulfuric acid, to obtain Compound 10-OHL

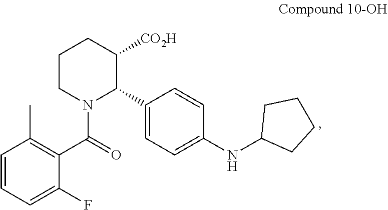 [SMILES: Cc1cccc(F)c1C(=O)N1CCC[C@H](C(=O)O)[C@@H]1c1ccc(NC2CCCC2)cc1]

and
converting compound 10-OH to Avacopan.
   44B. A process according to clause 43B, wherein Compound 10-OH is converted to Avacopan by reaction with 4-methyl-3-(trifluoromethyl)aniline optionally in the presence of a base, more preferably an alkali metal carbonate or an alkaline metal bicarbonate or an organic amine base; and more preferably wherein the base is triethylamine, sodium carbonate or potassium carbonate, particularly sodium carbonate or triethylamine.
   45B. A process according to clause 44B, wherein the reaction is carried out in the presence of an acid group activating coupling agent, preferably selected from the group consisting of, propylphosphonic anhydride (T3P), 1-ethyl-3-(3-dimethylaminopropyl)carbodiimide (EDC), and ethyl chloroformate.
   46B. A process according to clause 44B or clause 45B, wherein the reaction is carried out in a solvent, preferably a chlorinated hydrocarbon, an ester or a hydrocarbon; preferably wherein the chlorinated hydrocarbon is a chlorinated C₁ to C₆ hydrocarbon or chlorinated C₁ to C₃ hydrocarbon, and particularly dichloromethane; preferably wherein the hydrocarbon is a C₆ to C₁₀ preferably aromatic, hydrocarbon or a C₆ to C₈, preferably aromatic hydrocarbon, and particularly toluene; or preferably wherein the ester is a C₄ to C₈ ester, more preferably a C₂ to C₆ ester and particularly ethyl acetate.
   47B. A process according to any of clauses 44B to 46B, wherein the solvent is selected from the group consisting of: ethylacetate, dichloromethane or toluene.
   48B. A process according to any of clauses 38B to 42B, wherein step (d-ii) comprises reacting Compound 10 with 4-methyl-3-(trifluoromethyl)aniline, preferably in the presence of trimethyl aluminum.
   49B. A process according to any of clauses 38B to 42B, wherein step (d-ii) comprises converting Compound 10 to Compound 10-OH preferably according to the process of clause 43B; reacting Compound 10-OH with a chlorinating agent, to form Compound 10-C₁:

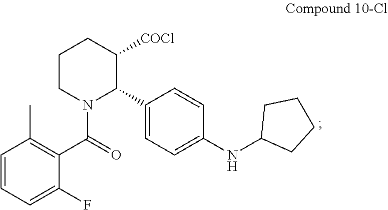 [SMILES: Cc1cccc(F)c1C(=O)N1CCC[C@H](C(=O)Cl)[C@@H]1c1ccc(NC2CCCC2)cc1]

and
reacting Compound 10-C₁ with 4-methyl-3-(trifluoromethyl)aniline to form Avacopan.
   50B. A process according to clause 49B, wherein the chlorinating agent is selected from thionyl chloride or oxalyl chloride, preferably thionyl chloride.
   51B. A process according to clause 49B or 50B, wherein the reaction of Compound 10-OH with a chlorinating agent is carried out in a solvent, preferably selected from: a chlorinated hydrocarbon, or a hydrocarbon; preferably wherein the chlorinated hydrocarbon is a chlorinated C₁ to C₆ hydrocarbon or a chlorinated C₁ to C₃ hydrocarbon, and particularly dichloromethane; or wherein the hydrocarbon is a C₆ to C₁₀ preferably aromatic, hydrocarbon or a C₆ to C₈, preferably aromatic hydrocarbon, and particularly toluene, and more preferably wherein the solvent is dichloromethane.
   52B. A process according to any of clauses 49B to 51B, wherein Compound 10-C₁ is reacted with 4-methyl-3-(trifluoromethyl)aniline directly as a solution from the chlorination reaction.
   53B. A process according to any of clauses 49B to 51B wherein Compound 10-C₁ is isolated from the chlorination reaction prior to reaction with 4-methyl-3-(trifluoromethyl)aniline.
   54B. A process according to any of clauses 49B to 53B, wherein the reaction of Compound 10-Cl with 4-methyl-3-(trifluoromethyl)aniline is carried out in the presence of a base, preferably an alkali metal carbonate or an alkaline metal bicarbonate or an organic amine base; and more preferably wherein the base is triethylamine, sodium carbonate or potassium carbonate, particularly triethylamine.
   55B. A process according to clause 36B or clause 37B, wherein Compound 5 is converted to Avacopan by a process comprising:
(a-iii) reacting Compound 5 with 2-fluoro-6-methylbenzoyl chloride to obtain Compound 7:

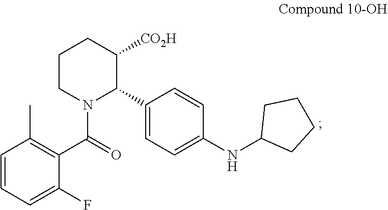 [SMILES: Cc1cccc(F)c1C(=O)N1CCC[C@H](C(=O)O)[C@@H]1c1ccc(NC2CCCC2)cc1]

(b-iii) converting Compound 7 to obtain Compound 8-OH:

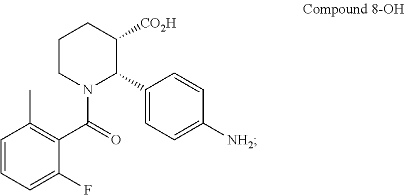 [SMILES: Cc1cccc(F)c1C(=O)N1CCC[C@H](C(=O)O)[C@@H]1c1ccc(N)cc1]

(c-iii) reacting Compound 8-OH with cyclopentanone to obtain Compound 10-OH:

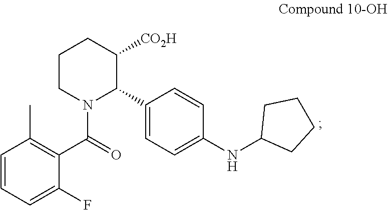 [SMILES: Cc1cccc(F)c1C(=O)N1CCC[C@H](C(=O)O)[C@@H]1c1ccc(NC2CCCC2)cc1]

(d-iii) reacting Compound 10-OH with 4-methyl-3-(trifluoromethyl)aniline to obtain Avacopan.
   56B. A process according to clause 55B, wherein step (a-iii) is carried out in a solvent comprising toluene and water, in the presence of a base, preferably an inorganic base, more preferably an alkali metal carbonate or an alkaline metal bicarbonate, and more preferably sodium carbonate or potassium carbonate, particularly sodium carbonate.
   57B. A process according to clause 55B or clause 56B, wherein step (b-iii) is carried out by hydrolysis using a mineral acid, preferably selected from hydrochloric acid, sulfuric acid or phosphoric acid, preferably hydrochloric acid or sulfuric acid.
   58B. A process according to clause 57B, wherein step (b-iii) is carried out in a solvent comprising water, preferably wherein the reaction is carried out at a temperature of: about 60° C. to about 110° C., about 70° C. to about 105° C., about 80° C. to about 100° C., or about 90° C. to about 95° C.
   59B. A process according to any of clauses 55B to 58B, wherein step (c-iii) is carried out in the presence of sodium triacetoxy borohydride, and optionally organic acid, preferably acetic acid.
   60B. A process according to clause 59B wherein step (c-iii) is carried out in the presence of a solvent, preferably a hydrocarbon such as a C₆ to C₁₀, preferably aromatic, hydrocarbon or a C₆ to C₈, preferably aromatic hydrocarbon, and particularly toluene.
   61B. A process for preparing Avacopan comprising:
(a-iv) hydrolysis of the ester of Compound 3:

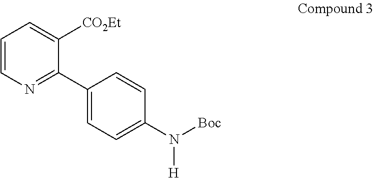 [SMILES: CCOC(=O)c1cccnc1-c1ccc(NC(=O)OC(C)(C)C)cc1]

to form Compound 29:

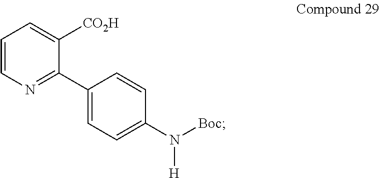 [SMILES: CC(C)(C)OC(=O)Nc1ccc(-c2ncccc2C(=O)O)cc1]

(b-iv) reacting Compound 29 with 4-methyl-3-(trifluoromethyl)aniline to form Compound 30:

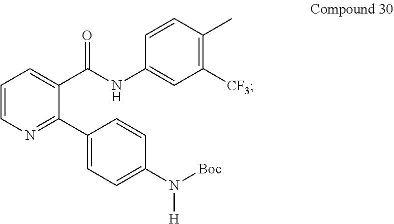 [SMILES: Cc1ccc(NC(=O)c2cccnc2-c2ccc(NC(=O)OC(C)(C)C)cc2)cc1C(F)(F)F]

(c-iv) hydrogenation of Compound 30 to form Compound 31:

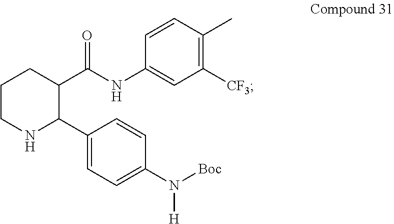 [SMILES: Cc1ccc(NC(=O)C2CCCNC2c2ccc(NC(=O)OC(C)(C)C)cc2)cc1C(F)(F)F]

(d-iv) optical resolution of Compound 32, preferably by diastereomeric salt separation using L-tolyl tartaric acid (L-DTTA), to form Compound 32:

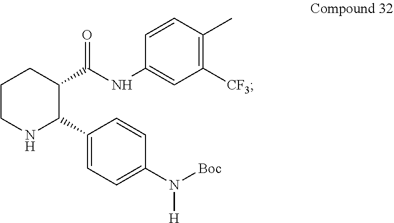

(e-iv) reaction of Compound 32 with 2-fluoro-6-methylbenzoyl chloride to form Compound 33:

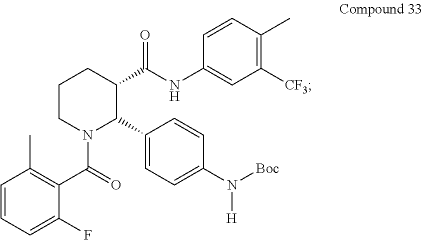

(f-iv) deprotecting Compound 33 to form Compound 34:

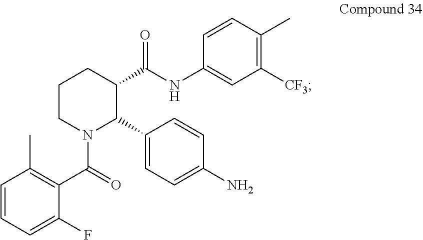

and
(g-iv) reacting Compound 34 with cyclopentanone.
   62B. A process according to clause 61B, wherein step (a-iv) comprises reacting Compound 3 with an alkali metal base, preferably sodium hydroxide or potassium hydroxide, and more preferably sodium hydroxide.
   63B. A process according to clause 62B, wherein the reaction is carried out in a solvent selected from one or more of an alcohol, water, and an ether; preferably wherein the alcohol, is a C₁ to C₆ alcohol, most preferably a C₁ to C₃ alcohol, and particularly methanol; and preferably wherein the ether is C₄ to C₈ ether or a C₄ to C₆ ether, and particularly tetrahydrofuran; more preferably wherein the solvent is a mixture of tetrahydrofuran, methanol and water.
   64B. A process according to clause 61B to 63B, wherein step (b-iv) comprise reacting Compound 29 with 4-methyl-3-(trifluoromethyl)aniline in the presence of an aprotic solvent, preferably selected from tetrahydrofuran, dimethyl formamide, dimethylsulfoxide, and more preferably dimethylformamide.
   65B. A process according to clause 64B, wherein step (b-iv) is carried out in the presence of a base, preferably an organic amine base, and more preferably triethylamine.
   66B. A process according to any of clauses 61B to 65B, wherein step (c-iv) comprises hydrogenating Compound 30 in the presence of a hydrogenation catalyst, preferably selected from palladium on carbon, platinum (II) oxide, Raney nickel in acetic acid, and more preferably wherein the hydrogenation catalyst is palladium on carbon.
   67B. A process according to clause 66B, wherein step (c-iv) is carried out in a solvent which is preferably acetic acid.
   68B. A process according to any of clauses 61B to 67B, wherein step (d-iv) comprises reaction of Compound 32 with L-DTTA, in a solvent, preferably wherein the solvent is a nitrile, preferably acetonitrile.
   69B. A process according to clause 68B, wherein Compound 32 is isolated by precipitation from the reaction mixture.
   70B. A process according to any of clauses 61B to 69B, wherein step (e-iv) comprises reacting Compound 32 with 2-fluoro-6-methylbenzoyl chloride in a solvent comprising toluene and water, in the presence of a base, preferably an inorganic base, more preferably an alkali metal carbonate or an alkaline metal bicarbonate, and more preferably sodium carbonate or potassium carbonate, particularly sodium carbonate.
   71B. A process according to any of clauses 61B to 70B, wherein step (f-iv) comprises reacting Compound 33 with a mineral acid, optionally an aqueous mineral acid, preferably wherein the acid is hydrochloric acid, and optionally in a solvent, preferably a hydrocarbon such as a C₆ to C₁₀ preferably aromatic, hydrocarbon or a C₆ to C₈, preferably aromatic hydrocarbon, and particularly toluene.
   72B. A process according to any of clauses 61B to 71B, wherein step (g-iv) comprises reacting Compound 34 with cyclopentanone in a solvent, preferably a hydrocarbon such as a C₆ to C₁₀ preferably aromatic, hydrocarbon or a C₆ to C₈, preferably aromatic hydrocarbon, and particularly toluene.
   73B. A process according to any preceding clause wherein compound 3 is prepared by a process comprising:
(A) reacting Compound 1:

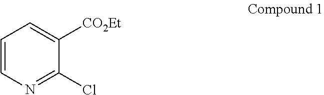 [SMILES: CCOC(=O)c1cccnc1Cl]

with Compound 2:

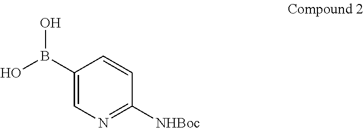 [SMILES: CC(C)(C)OC(=O)Nc1ccc(B(O)O)cn1]

to produce Compound 3:

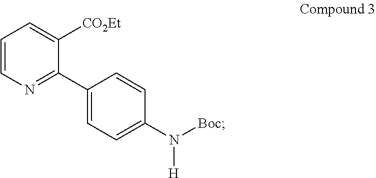 [SMILES: CCOC(=O)c1cccnc1-c1ccc(NC(=O)OC(C)(C)C)cc1]

and
(B) hydrogenation of Compound 3.
   74B. A process according to clause 73B wherein step (A) is carried out in the presence of a base, preferably an inorganic base, more preferably wherein the base is an alkali metal carbonate or alkali metal hydrogencarbonate; and particularly sodium carbonate, potassium carbonate, sodium hydrogencarbonate or potassium hydrogen carbonate, more particularly potassium carbonate.
   75B. A process according to clause 73B or clause 74B, wherein step (A) is carried out in the presence of a coupling agent, preferably palladium acetate/triphenyl phosphine, or bis(triphenylphosphine)palladium (II) dichloride.
   76B. A process according to any of clauses 73B to 75B, wherein step (A) is carried out in a solvent, preferably selected from the group consisting of a hydrocarbon, and water; preferably wherein the hydrocarbon is a C₆ to C₁₀ preferably aromatic, hydrocarbon or a C₆ to C₈, preferably aromatic hydrocarbon, and particularly toluene; and most preferably wherein the reaction is carried out in a solvent mixture comprising toluene and water.
   77B. A process according to any of clauses 73B to 76B, wherein step (B) is carried out by reacting Compound 3 with hydrogen in the presence of a hydrogenation catalyst, preferably wherein step (B) comprises hydrogenation of Compound 3 in the presence of a catalyst selected from palladium on carbon, platinum (II) oxide, Raney nickel in acetic acid, and more preferably wherein the hydrogenation catalyst is palladium on carbon or Raney nickel in acetic acid.
   78B. A process according to clause 77B, wherein step (B) is carried out in a solvent which is preferably acetic acid.
   79B. A process according to any of clauses 77B or 78B, wherein the reaction in step (B) is carried out at a temperature of: about 30° C. to about 90° C., about 40° C. to about 80° C., about 55° C. to about 75° C., or about 55° C. to about 65° C.
   80B. A process according to any of clauses 33B to 79B, further comprising combining the Avacopan with at least one pharmaceutically acceptable excipient to prepare a pharmaceutical composition.
   81B. Compound 4-Cl having the formula:

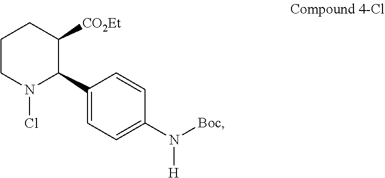 [SMILES: CCOC(=O)[C@@H]1CCCN(Cl)[C@@H]1c1ccc(NC(=O)OC(C)(C)C)cc1]

preferably wherein Compound 4-Cl is isolated, and more preferably wherein Compound 4-C₁ is in solid form.
   82B. Compound 5A having the formula:

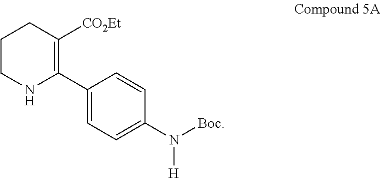 [SMILES: CCOC(=O)C1=C(c2ccc(NC(=O)OC(C)(C)C)cc2)NCCC1]

83B. Use of Compound 4-Cl or Compound 5A as defined in clause 81B or clause 82B, for the preparation of Avacopan.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### EXAMPLES

The following Examples 1-28 further serve to illustrate the optical resolution of racemic ethyl 2-(4-((tert-butoxycarbonyl)amino)phenyl)piperidine-3-carboxylate, as described in any aspect or embodiment of the disclosure, and the use of this process in the synthesis of Avacopan.

### Example 1: Process for ethyl 2-(4-((tert-butoxycarbonyl)amino)phenyl)nicotinate (Compound 3)

In a reactor equipped with condenser, charging tube, under nitrogen atmosphere, 30 gm of ethyl 2-chloronicotinate (“Compound 1”), 4-((tert-butoxycarbonyl)amino)phenyl)boronic acid (“Compound 2”) (37.8 g), Potassium carbonate (55.8 g), Toluene (450 ml), and water (150 ml) were charged. Nitrogen purged for 30-60 minutes at 20-30° C. Palladium acetate (0.36 g) and Triphenyl phosphine (0.85 g) were charged under nitrogen atmosphere. The reaction mass was heated 100-110° C., progress of reaction was monitored by TLC/HPLC, after completion of reaction, mass was cooled to 40-45° C. N-acetyl cysteine (4.5 g) added and stirred followed by filtration and layers separation. To organic layer solvent was distilled out under vacuum at 40-50° C. n-heptane (150 ml) was added, stirred, filtered and washed with n-heptane (30 ml) at 20-30° C. followed by drying under vacuum at 50-60° C. to afford ethyl 2-(4-((tert-butoxycarbonyl)amino)phenyl)nicotinate (Compound 3).

### Example 2: Process for ethyl 2-(4-((tert-butoxycarbonyl)amino)phenyl)nicotinate (Compound 3)

In a reactor equipped with condenser, charging tube, under nitrogen atmosphere, 30 gm of ethyl 2-chloronicotinate (Compound 1), 4-((tert-butoxycarbonyl)amino)phenyl)boronic acid (Compound 2) (37.8 g), Potassium carbonate (55.8 g), Toluene (450 ml), and water (150 ml) were charged. Nitrogen purged for 30-60 minutes at 20-30° C. Bis(triphenylphosphine)palladium(II) dichloride (1.1 g) was charged under nitrogen atmosphere. The reaction mass was heated 100-110° C., progress of reaction was monitored by TLC/HPLC, after completion of reaction, mass was cooled to 40-45° C. N-acetyl cysteine (4.5 g) added and stirred followed by filtration and layers separation. To organic layer solvent was distilled out under vacuum at 40-50° C. n-heptane (150 ml) was added, stirred, filtered and washed with n-heptane (30 ml) at 20-30° C. followed by drying under vacuum at 50-60° C. to afford ethyl 2-(4-((tert-butoxycarbonyl)amino)phenyl)nicotinate (Compound 3).

### Example 3: Process for ethyl 2-(4-((tert-butoxycarbonyl)amino)phenyl)piperidine-3-carboxylate (Compound 4)

In a hydrogenator reactor, under nitrogen atmosphere, 10 gm of ethyl 2-(4-((tert-butoxycarbonyl)amino)phenyl)nicotinate (Compound 3)., 10% Palladium carbon (1 g), and acetic acid (100 ml) were charged. Apply hydrogen pressure (10-15 kgcm−1) and mass was heated to 55-65° C., progress of reaction was monitored by TLC/HPLC. After completion of reaction, mass was filtered through hyflo followed by distillation of solvent. Methylene dichloride (20 ml) and aqueous saturated solution of sodium bicarbonate (20 ml), stirred for 30 minutes and phases were separated. Organic phase was distilled under vacuum at 40-50° C. to afford ethyl 2-(4-((tert-butoxycarbonyl)amino)phenyl)piperidine-3-carboxylate (Compound 4)

### Example 4: Process for ethyl 2-(4-((tert-butoxycarbonyl)amino)phenyl)piperidine-3-carboxylate (Compound 4)

In a hydrogenator reactor, under nitrogen atmosphere, 10 gm of ethyl 2-(4-((tert-butoxycarbonyl)amino)phenyl)nicotinate (Compound 3)., Raney Nickel (2 g), and acetic acid (100 ml) were charged. Apply hydrogen pressure (10-15 kgcm-1) and mass was heated to 55-65° C., progress of reaction was monitored by TLC/HPLC. After completion of reaction, mass was filtered through hyflo followed by distillation of solvent. Methylene dichloride (20 ml) and aqueous saturated solution of sodium bicarbonate (20 ml), stirred for 30 minutes and phases were separated. Organic phase was distilled under vacuum at 40-50° C. to afford ethyl 2-(4-((tert-butoxycarbonyl)amino)phenyl)piperidine-3-carboxylate (Compound 4)

### Example 5: Process for ethyl 2-(4-((tert-butoxycarbonyl)amino)phenyl)-1,4,5,6-tetrahydropyridine-3-carboxylate (Compound 5A)

In a reactor equipped with condenser, charging tube, under nitrogen atmosphere, 3 gm of ethyl (2S,3R)-2-(4-((tert-butoxycarbonyl)amino)phenyl)piperidine-3-carboxylate (Compound 4, S,R mixture, also referred to as Compound 5-ML) and Methylene dichloride (30 ml) were charged followed by charging of t-butanol (0.63 gm). The reaction mass was cooled to 0-10° C. Aq. solution of sodium hypochlorite (12.8 g) was added followed by addition of acetic acid (0.77 g), progress of reaction was monitored by TLC, after completion of reaction, water (15 ml) and methylene dichloride (15 ml) was added, stirred for 30 minute. Phases were separated, organic phase was distilled under vacuum until 8-10 volume of mass. (The mass is a solution containing ethyl (2S,3R)-2-(4-((tert-butoxycarbonyl)amino)phenyl)-1-chloropiperidine-3-carboxylate—“Compound 4-Cl”). 1,8-Diazabicyclo[5.4.0]undec-7-ene (DBU) (1.3 g) was charged and mass was stirred at 20-30° C., progress of reaction was monitored by TLC filtered, after completion of reaction, water (15 ml) was added and stirred. Phases were separated and organic phase was distilled under vacuum at 30-40° C. MTBE (15 ml) was added, stirred, filtered and washed with MTBE (6 ml) at 20-30° C. followed by dried under vacuum at 50-60° C. to afford ethyl 2-(4-((tert-butoxycarbonyl)amino)phenyl)-1,4,5,6-tetrahydropyridine-3-carboxylate (Compound 5A). Analysis for Compound 5A: ¹H NMR (DMSO-d₆, 500 MHz): δ 9.35 (1H, s), 7.39 (2H, ˜d, J˜9.0 Hz), 7.09 (2H, ˜d, J˜9.0 Hz), 6.32 (1H, t, J=3.5 Hz), 3.71 (2H, q, J=7.0 Hz), 3.19-3.11 (2H, m), 2.36 (2H, t, J=6.4 Hz), 1.74-1.70 (2H, m), 1.49 (9H, s), 0.84 (3H, t, J=7.0 Hz). ¹³C NMR (DMSO-d₆, 125 MHz): δ 167.6, 155.0, 152.7, 139.3, 133.4, 128.5 (2C), 117.0 (2C), 91.0, 79.0, 57.5, 41.1, 28.1 (3C), 23.4, 21.4, 14.1. IR (KBr): \tilde{v}=3396, 1705, 1666, 1534, 1239, 1160 cm⁻¹. HRMS (ESI) m/z: [M+H]⁺ Calcd for C₁₉H₂₇N₂O₄ 347.1966; Found 347.1956.

### Example 6: Process for ethyl 2-(4-((tert-butoxycarbonyl)amino)phenyl)-1,4,5,6-tetrahydropyridine-3-carboxylate (Compound 5A)

In a reactor equipped with condenser, charging tube, under nitrogen atmosphere, 2 gm of ethyl (2S,3R)-2-(4-((tert-butoxycarbonyl)amino)phenyl)piperidine-3-carboxylate (Compound 4, S,R mixture, also referred to as Compound 5-ML)) and Toluene (30 ml) were charged followed by slowly charging of Sodium dichloroisocyanurate (NaDCC) (0.8 gm) and water (4 ml). Progress of reaction was monitored by TLC, after completion of reaction, Methanol: water (1:1) 10 ml was added, stirred for 30 minute. Phases were separated (the organic phase contain ethyl (2S,3R)-2-(4-((tert-butoxycarbonyl)amino)phenyl)-1-chloropiperidine-3-carboxylate—“Compound 4-Cl”), 1,8-Diazabicyclo[5.4.0]undec-7-ene (DBU) (0.94 g) was charged and mass was stirred at 20-30° C., progress of reaction was monitored by TLC filtered, after completion of reaction, water (15 ml) was added and stirred. Phases were separated and organic phase was distill under vacuum at 30-40° C. MTBE (10 ml) was added, stirred, filtered and washed with MTBE (4 ml) at 20-30° C. followed by dried under vacuum at 50-60° C. to afford ethyl 2-(4-((tert-butoxycarbonyl)amino)phenyl)-1,4,5,6-tetrahydropyridine-3-carboxylate (Compound 5A).

### Example 7: Process for 2-(4-((tert-butoxycarbonyl)amino)phenyl)nicotinic acid (Compound 29)

In a reactor equipped with condenser, charging tube, under nitrogen atmosphere, 5 gm of ethyl 2-(4-((tert-butoxycarbonyl)amino)phenyl)nicotinate (Compound 3), Methanol (20 ml), Tetrahydrofuran (10 ml), water (10 ml) and sodium hydroxide (1.2 g) were charged. The reaction mass was heated 45-55° C., progress of reaction was monitored by TLC/HPLC, after completion of reaction, water (25 ml), acetic acid (5 ml) and ethyl acetate (50 ml) were added, stirred for 30 minute. Phases were separated, organic phase was washed with aqueous brine solution followed by distilled under vacuum at 40-50° C. Cyclohexane (25 ml) was added, stirred, filtered and washed with cyclohexane (10 ml) at 20-30° C. followed by drying under vacuum at 50-60° C. to afford 2-(4-((tert-butoxycarbonyl)amino)phenyl)nicotinic acid (Compound 29).

### Example 8: Process for tert-butyl (4-(3-((4-methyl-3-(trifluoromethyl) phenyl)carbamoyl) pyridin-2-yl)phenyl)carbamate (Compound 30)

In a reactor equipped with condenser, charging tube, under nitrogen atmosphere, 2 gm of 2-(4-((tert-butoxycarbonyl)amino)phenyl)nicotinic acid (Compound 29), 4-methyl-3-(trifluoromethyl)aniline (AVC-11) (1.11 g), Dimethyl formamide (10 ml) and Triethyl amine (1.6 g) were charged. The reaction mass was cooled to 5-10° C., followed by addition of HATU (2.5 g), progress of reaction was monitored by TLC/HPLC. After completion of reaction, water (20 ml), and ethyl acetate (20 ml) were added, stirred for 30 minute. Phases were separated, organic phase was washed with aqueous brine solution followed by distilled under vacuum at 40-50° C. Mixture of MTBE: Cyclohexane (1:1) (20 ml) added, stirred, filtered and washed with cyclohexane (10 ml) followed by drying under vacuum at 50-60° C. to afford tert-butyl (4-(3-((4-methyl-3-(trifluoromethyl)phenyl)carbamoyl)pyridin-2-yl)phenyl)carbamate (Compound 30).

### Example 9: Process for tert-butyl (4-(3-((4-methyl-3-(trifluoromethyl)phenyl) carbamoyl) piperidin-2-yl)phenyl)carbamate (Compound 31)

In a hydrogenator reactor, under nitrogen atmosphere, 2 gm of tert-butyl (4-(3-((4-methyl-3-(trifluoromethyl)phenyl)carbamoyl)pyridin-2-yl)phenyl)carbamate (Compound 30), 10% Palladium carbon (0.2 g), and acetic acid (50 ml) were charged. Apply hydrogen pressure (10-15 kgcm-1) and mass was heated to 55-65° C., progress of reaction was monitored by TLC/HPLC. After completion of reaction, mass was filtered through hyflo followed by distillation of solvent. Methylene dichloride (20 ml) and aqueous saturated solution of sodium bicarbonate (20 ml), stirred for 30 minutes and phases were separated. Organic phase was distilled under vacuum at 40-50° C. to afford tert-butyl (4-(3-((4-methyl-3-(trifluoromethyl)phenyl) carbamoyl) piperidin-2-yl)phenyl)carbamate (Compound 31).

### Example 10: Process for tert-butyl (4-((2R,3S)-3-((4-methyl-3-(trifluoromethyl)phenyl) carbamoyl)piperidin-2-yl)phenyl)carbamate (Compound 32)

In a reactor equipped with condenser, charging tube, under nitrogen atmosphere, 0.1 gm of tert-butyl (4-(3-((4-methyl-3-(trifluoromethyl)phenyl) carbamoyl)piperidin-2-yl)phenyl)carbamate (Compound 31), L-di-para-tolyl tartaric acid (0.08 g) and acetonitrile (1 ml) were charged. The reaction mass was stirred for 15 hrs at 20-30° C. Solid obtained was filtered and washed with acetonitrile (1 ml) followed by drying under vacuum at 50-60° C. to afford tert-butyl (4-((2R,3S)-3-((4-methyl-3-(trifluoromethyl)phenyl) carbamoyl)piperidin-2-yl)phenyl)carbamate (Compound 32).

### Example 11: Process for (2R,3S)-2-(4-aminophenyl)-1-(2-fluoro-6-methylbenzoyl)piperidine-3-carboxylic acid (Compound 8 OH)

In a reactor equipped with condenser, charging tube, under nitrogen atmosphere, ethyl (2R,3S)-2-(4-((tert-butoxycarbonyl)amino)phenyl)-1-(2-fluoro-6-methylbenzoyl)piperidine-3-carboxylate (Compound 7, 0.1 gm), Water (0.1 ml) and aqueous hydrochloric acid (0.5 ml) were charged. The reaction mass was heated to a temperature of about 90-95° C. and maintained for a period of about 2-4 hours at this temperature of about 90-95° C. After reaction completion, the mass was cooled down to a temperature of about 20-25° C. and the pH adjusted to 4-5 using aqueous sodium hydroxide solution followed by addition of ethyl acetate (1 ml), and stirring for 30 minute. The phases were separated, the organic phase was washed with aqueous brine solution followed by distilled under vacuum at a temperature of about 40-50° C. to give (2R,3S)-2-(4-aminophenyl)-1-(2-fluoro-6-methylbenzoyl)piperidine-3-carboxylic acid (Compound 8 OH).

### Example 12: Process for (2R,3S)-2-(4-aminophenyl)-1-(2-fluoro-6-methylbenzoyl)piperidine-3-carboxylic acid (Compound 8 OH)

In a reactor equipped with condenser, charging tube, under nitrogen atmosphere, ethyl (2R,3S)-2-(4-((tert-butoxycarbonyl)amino)phenyl)-1-(2-fluoro-6-methylbenzoyl)piperidine-3-carboxylate (Compound 7, 0.1 gm), and aqueous sulfuric acid (1 ml) were charged. The reaction mass was heated to a temperature of about 90-95° C. and maintained for a period of about 2-4 hours at a temperature of about 90-95° C. After reaction completion, mass was cooled to a temperature of about 20-25° C. and the pH adjusted to 4-5 using aqueous sodium hydroxide solution followed by addition of ethyl acetate (1 ml), and stirring for 30 minute. The phases were separated, the organic phase was washed with aqueous brine solution followed by distilled under vacuum at a temperature of about 40-50° C. to give (2R,3S)-2-(4-aminophenyl)-1-(2-fluoro-6-methylbenzoyl)piperidine-3-carboxylic acid (Compound 8 OH).

### Example 13: Process for (2R,3S)-2-(4-(cyclopentylamino)phenyl)-1-(2-fluoro-6-methylbenzoyl)piperidine-3-carboxylic acid (Compound 10 OH)

In a reactor equipped with condenser, charging tube, under nitrogen atmosphere, (2R,3S)-2-(4-aminophenyl)-1-(2-fluoro-6-methylbenzoyl)piperidine-3-carboxylic acid (Compound 8 OH, 1 gm), toluene (10 ml), acetic acid (0.17 g), Cyclopentanone (0.26 g) and sodium triacetoxy borohydride (1.2 g) were charged. The reaction mass was heated to a temperature of about 50-55° C. and maintained for a period of about 2-4 hours. After reaction completion, the mass was cooled to a temperature of about 20-25° C. followed by addition of acetic acid (2 ml), and stirring for 30 minute. The phases were separated followed by distillation under vacuum at 40-50° C. to give (2R,3S)-2-(4-(cyclopentylamino)phenyl)-1-(2-fluoro-6-methylbenzoyl)piperidine-3-carboxylic acid (Compound 10 OH).

### Example 14: Process for (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan)

In a reactor equipped with condenser, charging tube, under nitrogen atmosphere, (2R,3 S)-2-(4-(cyclopentylamino)phenyl)-1-(2-fluoro-6-methylbenzoyl)piperidine-3-carboxylic acid (Compound 10 OH, 0.1 gm), Ethyl acetate (2 ml), triethyl amine (47 mg), 4-methyl-3-(trifluoromethyl)aniline (54 mg) and T3P solution (50% in ethyl acetate (0.374 g) were charged. The reaction mass was heated to a temperature of about 55-60° C. and maintained for a period of about 8-12 hours. After reaction completion, mass was cooled to a temperature of about 20-25° C. followed by addition of sodium bicarbonate solution (3 ml), and stirring for 30 minute. The phases were separated followed by distillation under vacuum at a temperature of about 40-50° C. to give (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan).

### Example 15: Process for (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan)

In a reactor equipped with condenser, charging tube, under nitrogen atmosphere, (2R,3S)-2-(4-(cyclopentylamino)phenyl)-1-(2-fluoro-6-methylbenzoyl)piperidine-3-carboxylic acid (Compound 10 OH, 0.2 gm), dichloromethane (“MDC”, 2 ml), triethyl amine (0.1 g), 4-methyl-3-(trifluoromethyl)aniline (0.107 g) and EDC·HCl (0.225 g) were charged. The reaction mass was stirred to a temperature of about 20-25° C. and maintained for a period of about 4-6 hours. After reaction completion, sodium bicarbonate solution (3 ml) was added and stirred for 30 minute. The phases were separated followed by distillation under vacuum at a temperature of about 40-50° C. to give (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan).

### Example 16: Process for (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan)

In a reactor equipped with condenser, charging tube, under nitrogen atmosphere, (2R,3S)-2-(4-(cyclopentylamino)phenyl)-1-(2-fluoro-6-methylbenzoyl)piperidine-3-carboxylic acid (Compound 10 OH, 0.1 gm), dichloromethane (“MDC”, 1 ml), triethyl amine (59 mg), and ethyl chloroformate (38 mg) were charged at 0-10° C. The reaction mass was stirred to a temperature of about 0-10° C. and maintained for a period of about 1-2 hours followed by addition of 4-methyl-3-(trifluoromethyl)aniline (41 mg). The reaction mass was stirred to a temperature of about 20-25° C. and maintained for a period of about 2-4 hours. After reaction completion, water (1 ml) was added and stirred for 30 minute. The phases were separated followed by distillation under vacuum at a temperature of about 40-50° C. to give (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan).

### Example 17: Process for (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan)

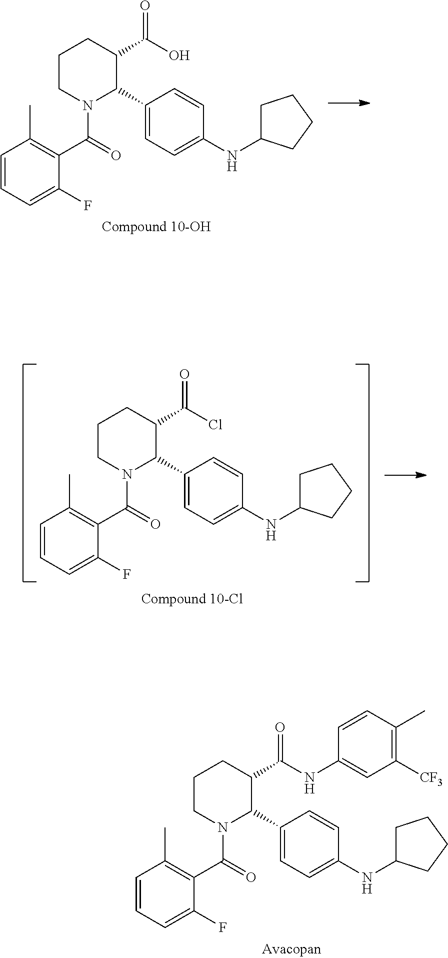

In a reactor equipped with condenser, charging tube, under nitrogen atmosphere, (2R,3S)-2-(4-(cyclopentylamino)phenyl)-1-(2-fluoro-6-methylbenzoyl)piperidine-3-carboxylic acid (Compound 10 OH, 0.5 gm), dichloromethane (“MDC”, 5 ml), Thionyl chloride (0.28 gm) were charged. The reaction mass was stirred to a temperature of about 25-35° C. and maintained for a period of about 2-4 hours. After reaction completion, solvent was distilled out under vacuum at 40-50° C. followed by addition of MDC (2.5 ml) to give compound-10-Cl solution. This solution was slowly added over a mixture of 4-methyl-3-(trifluoromethyl)aniline (0.21 gm), triethyl amine (0.35 gm), MDC (2.5 ml) at 20-30° C. and maintained for a period of about 2-4 hours. After reaction completion, water (5 ml) added and stirred for 30 minutes. The phases were separated followed by distillation under vacuum at a temperature of about 40-50° C. Ethanol (2.5 ml) and water (0.5 ml) was added followed by heating to 70-80° C. to give a clear solution. The mass was slowly cooled to 20-25° C. followed by filtration and washing to give 0.30 g ((2R, 3S)-2-(4-(cyclopentylamino) phenyl)-1-(2-fluoro-6-methylbenzoyl)-N-(4-methyl-3-(trifluoromethyl) phenyl)piperidine-3-carboxamide (Avacopan), Purity: >96%, Chiral purity: >99%.

### Example 18: Process for ethyl (2R,3S)-2-(4-((tert-butoxycarbonyl)amino)phenyl)-1-(2-fluoro-6-methylbenzoyl)piperidine-3-carboxylate (Compound 7)

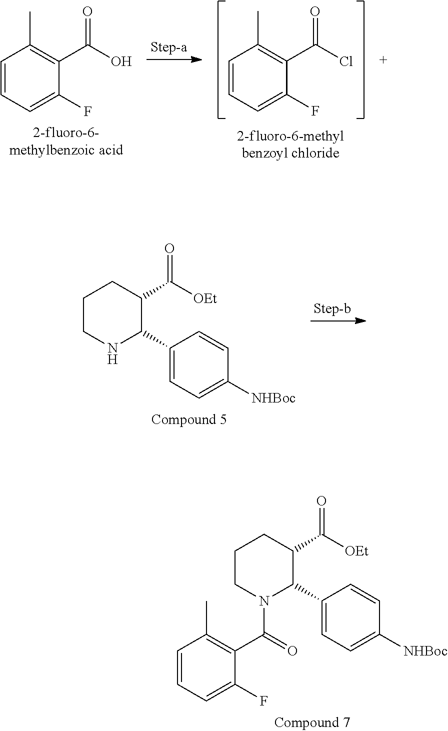

Step-a: In a reactor equipped with condenser, charging tube, under nitrogen atmosphere, 2-fluoro-6-methylbenzoic acid (46.4 gm), toluene (232 ml), Thionyl chloride (53.7 gm) and Dimethyl formamide (0.5 ml) were charged. The reaction mass was heated to 90-100° C. and stirred for 4-6 hrs at 90-100° C. After reaction completion mass was cooled to 20-30° C. and this toluene solution (2-fluoro-6-methyl benzoyl chloride) was used as such for step-b.

Step-b: In a reactor equipped with condenser, charging tube, under nitrogen atmosphere, ethyl (2R,3S)-2-(4-((tert-butoxycarbonyl)amino)phenyl)piperidine-3-carboxylate (Compound 5, 100 gm), Toluene (500 ml), water (1000 ml) and sodium carbonate (91 gm) were charged. The reaction mass was stirred and cooled to 10-20° C. Slowly added solution of 2-fluoro-6-methylbenzoyl chloride prepared in step-a at 10-20° C. The reaction mass temperature was raised to 20-30° C. and maintain for a period of about 1-2 hours. After reaction completion, ethyl acetate (500 ml) was added, stirred and layers were separated out. Solvents were distill out under vacuum at 50-60° C. followed by addition of n-heptane (300 ml) at 50-60° C. The mass was cooled and stirred for 2-4 hrs at 20-30° C. The solid was filtered off and washed with 100 ml of n-heptane to get ethyl (2R,3S)-2-(4-((tert-butoxycarbonyl)amino)phenyl)-1-(2-fluoro-6-methylbenzoyl)piperidine-3-carboxylate (compound 7).

### Example 19: Process for ethyl (2R,3S)-2-(4-aminophenyl)-1-(2-fluoro-6-methyl benzoyl) piperidine-3-carboxylate (Compound 8)

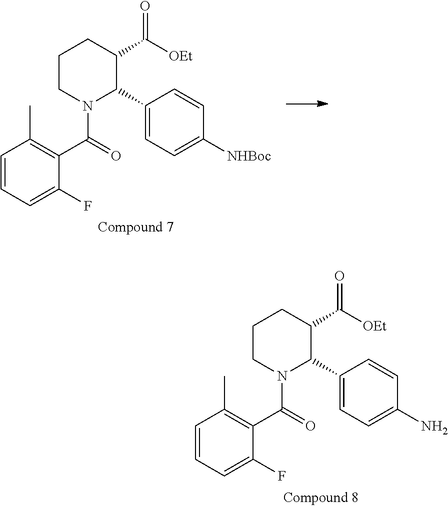

In a reactor equipped with condenser, charging tube, under nitrogen atmosphere, ethyl (2R,3S)-2-(4-((tert-butoxycarbonyl)amino)phenyl)-1-(2-fluoro-6-methylbenzoyl)piperidine-3-carboxylate (Compound 7, 10 gm), Toluene (50 ml) and aqueous concentrated hydrochloric acid (20 gm) were charged. The reaction mass was stirred at 15-25° C. and maintained for a period of about 2-4 hours at 15-25° C. After reaction completion, water (50 ml) was added followed by addition of aq. sodium hydroxide (20%) to adjust pH 6-7 at 20-30° C. The mass was stirred for 20-30 minutes at 20-30° C. The phases were separated followed by distillation under vacuum at a temperature of about 50-60° C. to get ethyl (2R,3S)-2-(4-aminophenyl)-1-(2-fluoro-6-methylbenzoyl)piperidine-3-carboxylate.

### Example 20: Purification of ethyl (2R,3S)-2-(4-aminophenyl)-1-(2-fluoro-6-methylbenzoyl) piperidine-3-carboxylate (Compound 8)

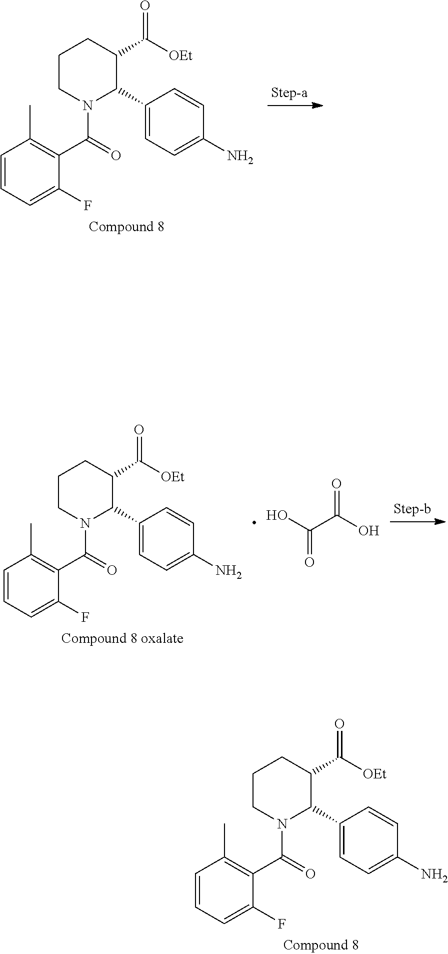

Step-a: In a reactor equipped with condenser, charging tube, under nitrogen atmosphere, ethyl (2R,3S)-2-(4-aminophenyl)-1-(2-fluoro-6-methylbenzoyl)piperidine-3-carboxylate (Compound 8, 10 gm), ethyl acetate (50 ml) and oxalic acid (3.5 gm) were charged. The reaction mass was heated to 50-60° C. and stirred for 1-2 hours at 50-60° C. The mass was cooled to 20-30° C. and maintain for a period of 2-3 hrs. The solid was filtered, washed with ethyl acetate to afford ethyl (2R,3S)-2-(4-aminophenyl)-1-(2-fluoro-6-methylbenzoyl)-piperidine-3-carboxylate oxalate (Compound 8 oxalate) yield 90-95% and having HPLC purity >99%.

Step-b: In a reactor equipped with condenser, charging tube, ethyl (2R,3S)-2-(4-aminophenyl)-1-(2-fluoro-6-methylbenzoyl)piperidine-3-carboxylate oxalate (Compound 8 oxalate, 10 gm), MDC (50 ml) and aqueous sodium bicarbonate saturated solution (50 ml) were charged. The reaction mass was stirred for 1-2 hours at 20-30° C. followed by phases separation. Solvent was distilled out under vacuum to get ethyl (2R,3S)-2-(4-aminophenyl)-1-(2-fluoro-6-methylbenzoyl)piperidine-3-carboxylate (Compound 8) having HPLC purity >99%.

### Example 21: Process for ethyl (2R, 3S)-2-(4-(cyclopentylamino)phenyl)-1-(2-fluoro-6-methylbenzoyl)piperidine-3-carboxylate (Compound 10)

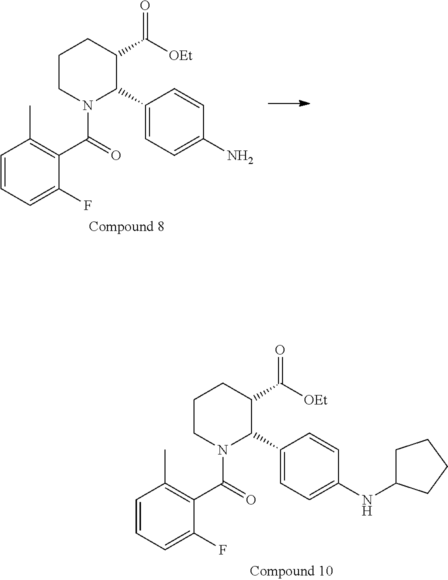

In a reactor equipped with condenser, charging tube, under nitrogen atmosphere, ethyl (2R,3S)-2-(4-aminophenyl)-1-(2-fluoro-6-methylbenzoyl)piperidine-3-carboxylate.

(Compound 8, 5 gm), Toluene (50 ml), Cyclopentanone (1.64 gm), p-toluene sulfonic acid (PTSA) (0.5 gm) and Hantzsch ester (7.25 gm) were charged. The reaction mass was stirred and heated to 70-75° C. and maintained for a period of about 2-4 hours. After reaction completion, reaction mass was cooled to 20-30° C. followed by addition of water (50 ml) and sodium bicarbonate (0.5 gm). The mass was stirred for 20-30 minutes at 20-30° C. The phases were separated followed by distillation under vacuum at a temperature of about 50-60° C. Methyl tertiary butyl ether (MTBE) (50 ml) was added and the mass slowly cooled to 20-25° C. followed by maintaining for 1-2 hours. The mass was filtered and washed with MTBE followed by purification using MTBE: toluene (4:0.5) mixture to give ethyl (2R,3S)-2-(4-(cyclopentyl-amino)phenyl)-1-(2-fluoro-6-methylbenzoyl)piperidine-3-carboxylate. (Compound 10).

### Example 22: Process for (2R,3S)-2-(4-(cyclopentylamino)phenyl)-1-(2-fluoro-6-methylbenzoyl) piperidine-3-carboxylic acid (Compound 10-OH)

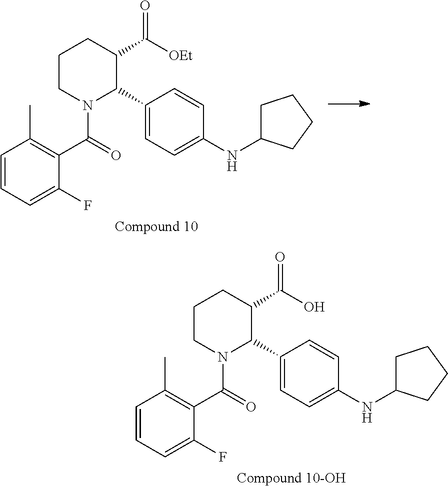

In a reactor equipped with condenser, charging tube, ethyl (2R,3S)-2-(4-(cyclopentylamino)phenyl)-1-(2-fluoro-6-methylbenzoyl)piperidine-3-carboxylate (Compound 10, 5 gm), and 17% aqueous Sulfuric acid (50 ml) were charged. The reaction mass was stirred and heated to 95-100° C. The reaction mass was stirred for 6-7 hours at 95-100° C. After reaction completion, the mass was cooled to 20-30° C. followed by addition of water (50 ml). The pH of reaction mass was adjusted to 12-13 by adding aq. NaOH solution followed by addition of Methyl tertiary butyl ether. The mass was stirred and phases were separated at 20-30° C. To aqueous layer toluene (50 ml) was added followed by pH adjustment to 4-5 using aqueous sulfuric acid. The mass was stirred and phases were separated followed by distillation solvent under vacuum to get (2R,3S)-2-(4-(cyclopentylamino)phenyl)-1-(2-fluoro-6-methylbenzoyl)piperidine-3-carboxylic acid (Compound 10-OH).

### Example 23: Process for (2R,3S)-2-(4-(cyclopentylamino)phenyl)-1-(2-fluoro-6-methyl benzoyl)-N-(4-methyl-3-(trifluoromethyl)phenyl)piperidine-3-carboxamide (Avacopan)

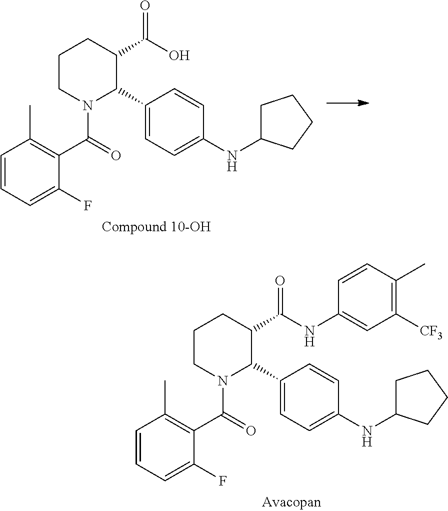

In a reactor equipped with condenser, charging tube, under nitrogen atmosphere, (2R,3S)-2-(4-(cyclopentylamino)phenyl)-1-(2-fluoro-6-methylbenzoyl)piperidine-3-carboxylic acid

(Compound 10-OH, 50 gm), Toluene (500 ml), Sodium carbonate (18.7 gm) and 4-methyl-3-(trifluoromethyl)aniline (21.6 gm) were charged. The reaction mass cooled reaction mass 10-20° C. followed by addition of methane sulfonyl chloride (Mscl) (17.5 gm) at 10-20° C. The reaction mass was stirred for 6-12 hrs at 20-30° C. After completion of reaction, water (250 ml) was added and mass was stirred at 20-30° C. The phases were separated out followed by distillation under vacuum at a temperature of about 50-60° C. Methanol (200 ml), water (35 ml) were added and the mass was stirred at 50-60° C. for 1 hr. The mass was cooled to 20-30° C., the slurry was stirred for 2-4 hrs. The solid was filtered off and washed with 50 ml portion of 8:2 methanol/water to get crude (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan). To crude compound methanol (200 ml) and water (20 ml) were charged and mass was heated to 55-65° C. and maintained for 1 hr at 55-65° C. The reaction mass was cooled to 20-30° C. and stirred for 2-4 hrs. The solid was filtered and washed with 50 ml portion of 9:1 methanol: water to get (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (Avacopan) as off-white crystal with yield of 80% meeting ICH quality.

### Example 24: Process for ethyl 2-(4-((tert-butoxycarbonyl)amino)phenyl)piperidine-3-carboxylate (Compound 4)

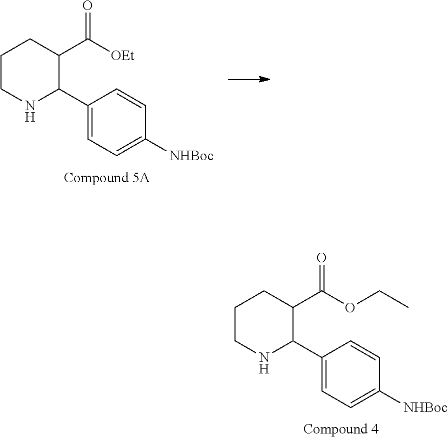 [SMILES: CCOC(=O)C1CCCNC1c1ccc(NC(=O)OC(C)(C)C)cc1 ; CCOC(=O)C1CCCNC1c1ccc(NC(=O)OC(C)(C)C)cc1]

In a hydrogenator reactor, under nitrogen atmosphere, 300 gm of ethyl 2-(4-((tert-butoxycarbonyl)amino)phenyl)-1,4,5,6-tetrahydropyridine-3-carboxylate (Compound 5A) 10% Palladium carbon (30 g), isopropyl alcohol (1500 ml) and acetic acid (300 ml) were charged. Apply hydrogen pressure (10-15 kg/cm2) and mass was heated to 55-65° C., progress of reaction was monitored. After completion of reaction, mass was filtered through hyflo followed by distillation of solvent. Toluene (600 ml) was added and pH 6.5-7.5 was adjusted by adding aqueous saturated solution of sodium carbonate. The mass was stirred for 30 minutes and phases were separated. Organic phase was distilled under vacuum at 40-50° C. to afford ethyl 2-(4-((tert-butoxycarbonyl)amino)phenyl)piperidine-3-carboxylate.

### Example 25: Process for ethyl (2R,3S)-2-(4-((tert-butoxycarbonyl)amino)phenyl)-piperidine-3-carboxylate (Compound 5)

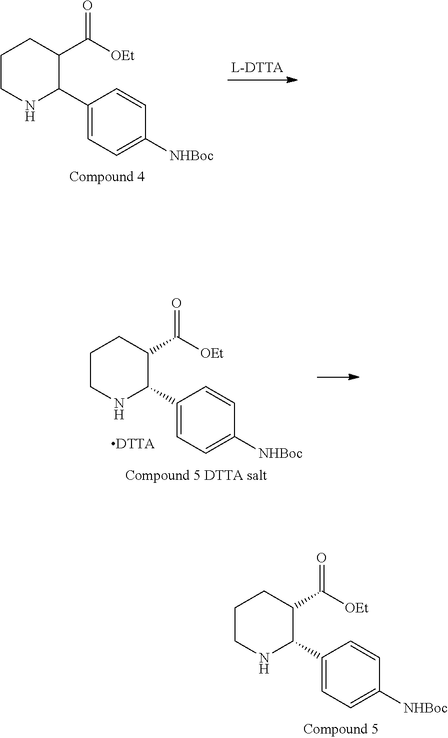

Step-a: In a reactor equipped with condenser, charging tube, under nitrogen atmosphere, ethyl 2-(4-((tert-butoxycarbonyl)amino)phenyl)piperidine-3-carboxylate (Compound 4, 100 gm), Ethanol (500 ml), and Di-p-tolyl-L-tartaric acid (110 gm) were charged. The reaction mass was stirred and heated to 50-55° C. Solvent was distilled out under vacuum at 50-55° C. followed by addition of Isopropyl acetate (1000 ml). Mass was heated to 70-75° C. followed by slow addition of MTBE (1000 ml). The mass cooled and stirred for 10-12 hrs at 20-30° C. The solid was filtered off and washed with MTBE (100 ml) followed by purification using THF: MTBE to get ethyl (2R,3S)-2-(4-((tert-butoxycarbonyl)amino)phenyl)piperidine-3-carboxylate DTTA salt (Compound 5 DTTA salt).

Step-b: In a reactor equipped with condenser, charging tube, under nitrogen atmosphere, ethyl (2R,3S)-2-(4-((tert-butoxycarbonyl)amino)phenyl)piperidine-3-carboxylate DTTA (Compound 5 salt, 100 g), MDC (1000 ml). The reaction mass was stirred and aqueous sodium bicarbonate solution (1000 ml) was charged. The mass was stir followed by layers separation. Solvent was distilled out under vacuum at 40-50° C. get ethyl (2R,3S)-2-(4-((tert-butoxycarbonyl)amino)phenyl)piperidine-3-carboxylate (Compound 5).

### Example 26: Process for ethyl (2R,3S)-2-(4-((tert-butoxycarbonyl)amino)phenyl)-piperidine-3-carboxylate (Compound 5)

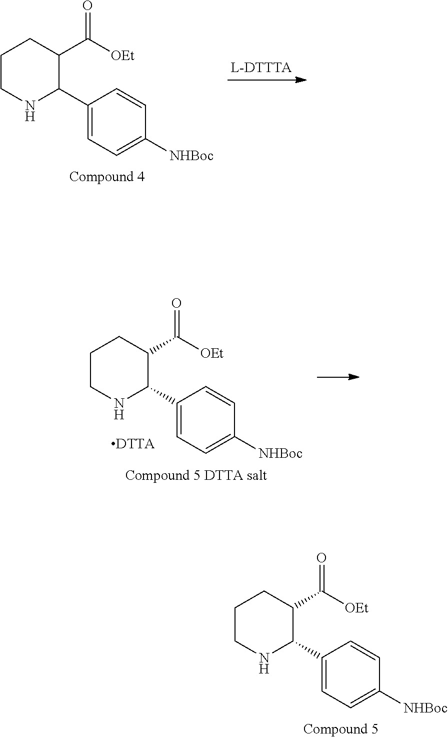

Step-a: In a reactor equipped with condenser, charging tube, under nitrogen atmosphere, ethyl 2-(4-((tert-butoxycarbonyl)amino)phenyl)piperidine-3-carboxylate (Compound 4, 100 gm), Ethanol (500 ml), and Di-p-tolyl-L-tartaric acid (110 gm) were charged. The reaction mass was stirred and heated to 50-55° C. Solvent was distilled out under vacuum at 50-55° C. followed by addition of Isopropyl acetate (1000 ml). Mass was heated to 70-75° C. followed by slow addition of MTBE (1000 ml). The mass cooled and stirred for 10-12 hrs at 20-30° C. The solid was filtered off and washed with MTBE (100 ml) followed by purification using THF: MTBE to get solid ethyl (2R,3S)-2-(4-((tert-butoxycarbonyl)amino)phenyl)piperidine-3-carboxylate DTTA salt (Compound 5 L-DTTA salt) and filtrate concentrated to get oil of ethyl (2S,3R)-2-(4-((tert-butoxycarbonyl)amino)phenyl)piperidine-3-carboxylate DTTA salt (Compound 5 ML DTTA).

Step-b: In a reactor equipped with condenser, charging tube, under nitrogen atmosphere, ethyl (2R,3S)-2-(4-((tert-butoxycarbonyl)amino)phenyl)piperidine-3-carboxylate DTTA (Compound 5 L-DTTA, 100 g) and Methylene dichloride (1000 ml). The reaction mass was stirred and aqueous sodium bicarbonate solution (1000 ml) was charged. The mass was stir followed by layers separation. Solvent was distilled out under vacuum at 40-50° C. get ethyl (2R,3S)-2-(4-((tert-butoxycarbonyl)amino)phenyl)piperidine-3-carboxylate (Compound 5).

### Example 27: Process for ethyl (2S,3R)-2-(4-((tert-butoxycarbonyl)amino)phenyl)-piperidine-3-carboxylate (Compound 5 ML)

In a reactor equipped with condenser, charging tube, under nitrogen atmosphere, 10 gm L-DTTA salt of ethyl (2S,3R)-2-(4-((tert-butoxycarbonyl)amino)phenyl)piperidine-3-carboxylate (Compound 5 ML DTTA) and Methylene dichloride (100 ml) were charged followed by addition of aqueous sodium bicarbonate solution. The reaction mass was stirred for 30 minute, phases were separated, organic phase was distilled under vacuum at 35-45° C. to get ethyl (2S,3R)-2-(4-((tert-butoxycarbonyl)amino)phenyl)piperidine-3-carboxylate (Compound 5 ML).

### Example 28: Process for (2R,3S)-2-(4-(cyclopentylamino)phenyl)-1-(2-fluoro-6-methyl benzoyl)-N-(4-methyl-3-(trifluoromethyl)phenyl)piperidine-3-carboxamide (Avacopan) amorphous

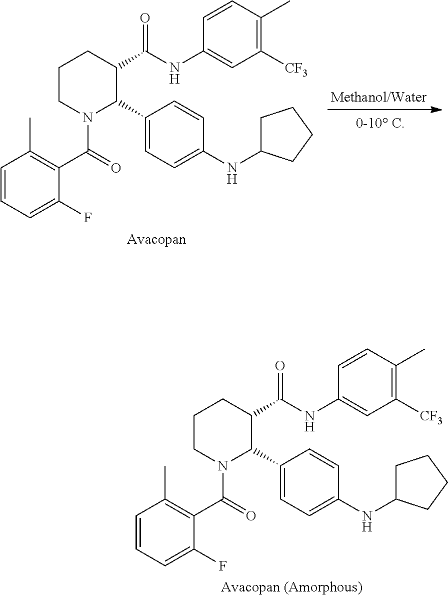

In a clean and dry reactor equipped with condenser, charging tube, (2R,3S)-2-(4-(cyclopentylamino)phenyl)-1-(2-fluoro-6-methylbenzoyl)-N-(4-methyl-3-(trifluoromethyl)phenyl)piperidine-3-carboxamide (25 gm) and Methanol (750 ml) were charged. The mass was heated to 45-55° C. followed by filtration through hyflo bed to get a clear solution. In a separate reactor charge DM Water (2.5 L) and cooled to 0-5° C. Add slowly above clear methanol solution to DM water at 0-5° C. Stirred and maintained for 2 hours at 0-10° C. followed by filtration and drying to give (2R,3S)-2-(4-(cyclopentylamino)phenyl)-1-(2-fluoro-6-methylbenzoyl)-N-(4-methyl-3-(trifluoromethyl)phenyl)piperidine-3-carboxamide in amorphous form. A PXRD figure is shown in FIG. 1.

### Powder X-Ray Diffraction (XRD) Method

X-ray diffraction was performed on X-Ray powder diffractometer:

Bruker D8 Advance; CuK_radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.
Divergence slit: V20

The following Examples 29-39 illustrate the enzymatic kinetic resolution procedure for racemic ethyl 2-(4-((tert-butoxycarbonyl)amino)phenyl)piperidine-3-carboxylate, as described herein, and the use of this process in the synthesis of Avacopan.

Preparation of the starting compound, Compound 4 may be done by any process disclosed in the literature. Alternatively, and for example, it may be prepared by the process disclosed in Examples 1-4.

Preparation of the Avacopan intermediates, Compound 5 and Compound 5a may be done using an imine reductase enzyme as the main enzyme for oxidative kinetic resolution of Compound 4. Such imine reductase enzyme can be purchased (for example, PROZOMIX PRO-IRED-128) or prepared (TPW-T164 was prepared as described herein below in Example 39). This process may be done using a second enzyme for cofactor regeneration (NADP⁺ reduction to NADPH), such as alcohol dehydrogenase from Lactobacillus brevis. Such an enzyme, for example, is LbADH, TPW-T004, Uniprot: Q03TF9; which was prepared as described herein below in Example 39.

### Example 29: Process for ethyl 2-(4-((tert-butoxycarbonyl)amino)phenyl)nicotinate (Compound 3)

In a reactor equipped with condenser, charging tube, under nitrogen atmosphere, 30 grams of ethyl 2-chloronicotinate (“Compound 1”), 4-((tert-butoxycarbonyl)amino)phenyl)boronic acid (“Compound 2”) (37.8 grams), Potassium carbonate (55.8 grams), Toluene (450 ml), and water (150 ml) were charged. Nitrogen purged for 30-60 minutes at 20-30° C. Palladium acetate (0.36 grams) and Triphenyl phosphine (0.85 grams) were charged under nitrogen atmosphere. The reaction mass was heated 100-110° C., progress of reaction was monitored by TLC/HPLC, after completion of reaction, mass was cooled to 40-45° C. N-acetyl cysteine (4.5 grams) added and stirred followed by filtration and layers separation. To organic layer solvent was distilled out under vacuum at 40-50° C. n-heptane (150 ml) was added, stirred, filtered and washed with n-heptane (30 ml) at 20-30° C. followed by drying under vacuum at 50-60° C. to afford ethyl 2-(4-((tert-butoxycarbonyl)amino)phenyl)nicotinate (Compound 3).

### Example 30: Process for ethyl 2-(4-((tert-butoxycarbonyl)amino)phenyl)nicotinate (Compound 3)

In a reactor equipped with condenser, charging tube, under nitrogen atmosphere, 30 grams of ethyl 2-chloronicotinate (Compound 1), 4-((tert-butoxycarbonyl)amino)phenyl)boronic acid (Compound 2) (37.8 grams), Potassium carbonate (55.8 grams), Toluene (450 ml), and water (150 ml) were charged. Nitrogen purged for 30-60 minutes at 20-30° C. Bis(triphenylphosphine)palladium(II) dichloride (1.1 grams) was charged under nitrogen atmosphere. The reaction mass was heated 100-110° C., progress of reaction was monitored by TLC/HPLC, after completion of reaction, mass was cooled to 40-45° C. N-acetyl cysteine (4.5 grams) added and stirred followed by filtration and layers separation. To organic layer solvent was distilled out under vacuum at 40-50° C. n-heptane (150 ml) was added, stirred, filtered and washed with n-heptane (30 ml) at 20-30° C. followed by drying under vacuum at 50-60° C. to afford ethyl 2-(4-((tert-butoxycarbonyl)amino)phenyl)nicotinate (Compound 3).

### Example 31: Process for ethyl 2-(4-((tert-butoxycarbonyl)amino)phenyl)piperidine-3-carboxylate (Compound 4)

In a hydrogenator reactor, under nitrogen atmosphere, 10 grams of ethyl 2-(4-((tert-butoxycarbonyl)amino)phenyl)nicotinate (Compound 3), 10% Palladium carbon (1 gram), and acetic acid (100 ml) were charged. Apply hydrogen pressure (10-15 kg/cm² or 10-15 bar) and mass was heated to 55-65° C., progress of reaction was monitored by TLC/HPLC. After completion of reaction, mass was filtered through hyflo followed by distillation of solvent. Methylene dichloride (20 ml) and aqueous saturated solution of sodium bicarbonate (20 ml) were stirred for 30 minutes and phases were separated. Organic phase was distilled under vacuum at 40-50° C. to afford ethyl 2-(4-((tert-butoxycarbonyl)amino)phenyl)piperidine-3-carboxylate (Compound 4).

### Example 32: Process for ethyl 2-(4-((tert-butoxycarbonyl)amino)phenyl)piperidine-3-carboxylate (Compound 4)

In a hydrogenator reactor, under nitrogen atmosphere, 10 grams of ethyl 2-(4-((tert-butoxycarbonyl)amino)phenyl)nicotinate (Compound 3), Raney Nickel (2 grams), and acetic acid (100 ml) were charged. Apply hydrogen pressure (10-15 kg/cm² or 10-15 bar) and mass was heated to 55-65° C., progress of reaction was monitored by TLC/HPLC. After completion of reaction, mass was filtered through hyflo followed by distillation of solvent. Methylene dichloride (20 ml) and aqueous saturated solution of sodium bicarbonate (20 ml) were stirred for 30 minutes and phases were separated. Organic phase was distilled under vacuum at 40-50° C. to afford ethyl 2-(4-((tert-butoxycarbonyl)amino)phenyl)piperidine-3-carboxylate (Compound 4).

### Example 33: Kinetic resolution of cis-Ethyl 2-(4-((tert-butoxycarbonyl) amino) phenyl) piperidine-3-carboxylate (Compound 4)

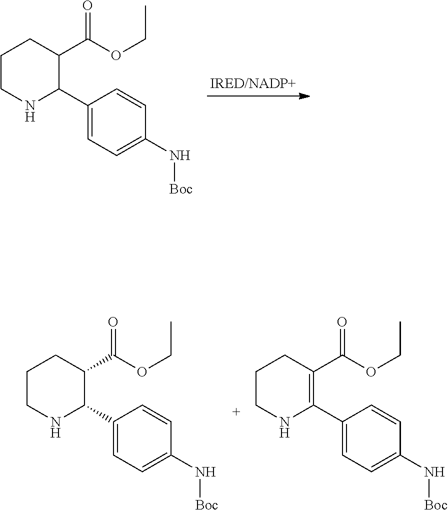 [SMILES: CCOC(=O)C1CCCNC1c1ccc(NC(=O)OC(C)(C)C)cc1 ; CCOC(=O)[C@H]1CCCN[C@H]1c1ccc(NC(=O)OC(C)(C)C)cc1 ; CCOC(=O)C1=C(c2ccc(NC(=O)OC(C)(C)C)cc2)NCCC1]

Step a: β-Nicotinamide adenine dinucleotide phosphate disodium salt (NADP+Na, 2.00 grams, 2.61 mmol) and NADPH oxidase enzyme (9.00 grams, Pro-Nox-001 purchased from Prozomix) were dissolved in 2250 ml 0.1 M TRIS buffer (TRIS buffer pre-prepared by dissolving 63.76 grams Tris(hydroxymethyl)aminomethane (0.5 mol) in 5000 ml deionized (DI) water and the pH was adjusted to 8.5 with 18 m/V % hydrochloric acid solution).

Step b: Imine reductase enzyme (9 grams) was dissolved in 2025 ml 0.1 M TRIS buffer and combined with the cofactor solution of Step a. The mixture was saturated by air.

cis-Ethyl 2-(4-((tert-butoxycarbonyl)amino)phenyl)piperidine-3-carboxylate (Compound 4, 9.00 grams, 25.83 mmol) was dissolved in 225 ml DMSO by stirring at room temperature for 30 minutes and then added to the enzyme-cofactor solution. The obtained reaction mixture was stirred at 24° C. under continuous slow air-flow and monitored by HPLC. After the reaction completion (at near 50% decrease of starting material) the resulting products were isolated by pH-dependent extraction with Toluene.

The pH of the reaction mixture was adjusted to 3.2 and mixed with 6 L Toluene. 50 grams Na₂SO₄ and 300 grams Hyflo® Super Cel® were added to the mixture. Filter aids were filtered out, then phases were separated and the extraction was repeated with 2 L Toluene. The combined organic layers were evaporated to dryness to get 4.34 grams (43%) ethyl 2-(4-((tert-butoxycarbonyl)amino)phenyl)-1,4,5,6-tetrahydropyridine-3-carboxylate (Compound 5A) with 96.55 Area % HPLC purity. The pH of the twice extracted aqueous phase was adjusted to 8.2 and extracted with 6 L and 2 L Toluene. The combined organic layers were evaporated to dryness to get 4.72 grams (41%) ethyl (2R,3S)-2-(4-((tert-butoxycarbonyl)amino)-phenyl)piperidine-3-carboxylate (Compound 5) with 95.96 Area % HPLC purity and 98.09% ee.

The Imine reductase enzyme which may be used in thus procedure can be PRO-IRED-104, PRO-IRED-107, PRO-IRED-109, PRO-IRED-114 and PRO-IRED-128.

### Example 34: Kinetic resolution of cis-Ethyl 2-(4-((tert-butoxycarbonyl) amino) phenyl) piperidine-3-carboxylate (Compound 4)

Kinetic resolution of cis-Ethyl 2-(4-((tert-butoxycarbonyl)amino)phenyl) piperidine-3-carboxylate can be implemented in the presence of Alcohol dehydrogenase or Ketoreductase cofactor regenerating enzymes in presence of Acetone co-substrate.

Step a: β-Nicotinamide adenine dinucleotide phosphate disodium salt (NADP+Na, 0.58 grams, 0.5 mM), PRO-IRED-128 imine reductase enzyme (3.75 grams, 25 w/w %) and alcohol dehydrogenase enzyme from Lactobacillus brevis (0.75 grams, 5w/w %) were dissolved in 1380 ml 0.1 M KH2PO4 buffer (Buffer solution prepared by dissolving 21.49 grams KH2PO4 in 1500 ml deionized (DI) water and the pH was adjusted to 7.5 with 18 m/V % hydrochloric acid solution). 45 ml Acetone (3 v %) was charged to the previously prepared enzyme-cofactor solution (Step a). 15 grams (43 mmol) cis-Ethyl 2-(4-((tert-butoxycarbonyl) amino)phenyl)piperidine-3-carboxylate (Compound 4) was dissolved in 75 ml (5 v %) DMSO and charged to the enzyme solution. The reaction mixture was stirred at 24° C. The reaction completion was monitored by HPLC. The resulting products were isolated separately by filtration and extraction with 2-Me-THF. The reaction mixture was filtered three times on a filter layer having pore size 5 um until the filtrate became clear. The filtered crude solid (7.32 grams) was stirred in 740 ml Acetone at RT. The undissolved solid material filtered out, and the filtrate was evaporated to dryness at reduced pressure in order to get 6.1 grams (41%) ethyl 2-(4-((tert-butoxycarbonyl)amino)phenyl)-1,4,5,6-tetrahydropyridine-3-carboxylate (Compound 5A) with 93.39 Area % HPLC purity.

30 grams Na₂SO₄ and 150 grams Hyflo® Super Cel® were added to the filtered reaction mixture and extracted two times with 2250 ml and 1500 ml (1.5 and 1.0 vol eq) 2-Me-THF. The combined organic layers were dried on Na₂SO₄, filtered and evaporated to dryness at reduced pressure in order to get 8.2 grams (42%) ethyl (2R,3S)-2-(4-((tert-butoxycarbonyl)amino)phenyl)piperidine-3-carboxylate (Compound 5) with 97.9 Area % HPLC purity and 97.94% ee.

Suggested Alcohol dehydrogenases are: Pro-KRED-067 purchased from Prozomix, or Alcohol dehydrogenase from Lactobacillus brevis (LbADH).

The Imine reductase enzyme which may be used in thus procedure can be PRO-IRED-104, PRO-IRED-107, PRO-IRED-109, PRO-IRED-114 and PRO-IRED-128.

### Example 35: Kinetic resolution of cis-Ethyl 2-(4-((tert-butoxycarbonyl) amino) phenyl) piperidine-3-carboxylate (Compound 4)

Step a: β-Nicotinamide adenine dinucleotide phosphate disodium salt (NADP+Na, 1.67 grams, 0.5 mM), TPW-T164 Imine reductase enzyme (10.00 grams, 25 w/w %) and Alcohol dehydrogenase enzyme from Lactobacillus brevis (TPW-T004, 2.00 grams, 5w/w %) were dissolved in 4000 ml 0.1 M potassium phosphate buffer (Buffer solution prepared by dissolving 64.07 grams K₂HPO₄ and 17.99 grams KH2PO4 in 5000 ml deionized (DI) water and the pH was adjusted to 7.5 with 23 m/V % NaOH solution).

Step b: 120 ml Acetone (3 v %) was charged to the previously prepared enzyme-cofactor solution prepared in Step a. 40 grams (114.8 mmol) cis-Ethyl 2-(4-((tert-butoxycarbonyl) amino)phenyl)piperidine-3-carboxylate (Compound 4) was dissolved in 200 ml (5 v %) DMSO and charged to the enzyme solution. The reaction mixture was stirred at 24° C. The reaction completion was monitored by HPLC. The resulting products were isolated separately by filtration and extraction with Ethyl-acetate. The reaction mixture was mixed with 5 grams Hyflo® Super Cel®, then filtered three times on a filter layer having pore size 5 um until the filtrate became clear. The solid residue was stirred with 200 ml Acetone at 30° C. for 30 minutes. The filter aid was filtered out, and the filtrate was evaporated to ¼ volume at reduced pressure and precipitated by the addition of 250 ml pre-cooled water (4° C.). Precipitated solid material was filtered out and dried in vacuum at 40° C. to get 14.93 grams (Yield: 37%) ethyl 2-(4-((tert-butoxycarbonyl)amino)phenyl)-1,4,5,6-tetrahydropyridine-3-carboxylate (Compound 5A) with 99.94 Area % HPLC purity.

10 grams Na₂SO₄ and 50 grams Hyflo® Super Cel® were added to the clear filtrate of the reaction mixture and extracted two times with 3200 ml (0.75 vol eq) EtOAc. The combined organic layers were dried on Na₂SO₄, filtered and evaporated to dryness at reduced pressure in order to get 13.83 grams (Yield: 34.58%) ethyl (2R,3S)-2-(4-((tert-butoxycarbonyl)amino)phenyl)piperidine-3-carboxylate (Compound 5) with 97.86 Area % HPLC purity and 99.71% ee.

### Example 36: Kinetic resolution of cis-Ethyl 2-(4-((tert-butoxycarbonyl) amino) phenyl) piperidine-3-carboxylate (Compound 4)

Step a: β-Nicotinamide adenine dinucleotide phosphate disodium salt (NADP+Na, 30 grams, 0.5 mM), TPW-T164 Imine reductase enzyme (250 grams, 25 w/w %) and Alcohol dehydrogenase enzyme from Lactobacillus brevis (TPW-T004, 50 grams, 5w/w %) were dissolved in 100 L 0.1 M KH2PO4 buffer (Buffer solution prepared by dissolving 1.8 kg KH2PO4 in 125 L deionized (DI) water and the pH was adjusted to 7.5 with 23 m/V % NaOH solution).

Step b: 3 L Acetone (3 v %) was charged to the previously prepared enzyme-cofactor solution prepared in step a). 1000 grams (2.87 mol) cis-Ethyl 2-(4-((tert-butoxycarbonyl) amino)phenyl)piperidine-3-carboxylate (Compound 4) was dissolved in 5 L (5 v %) DMSO and charged to the enzyme solution. The reaction mixture was stirred at 24° C. The reaction completion was monitored by HPLC. The resulting products were isolated separately by filtration and extraction with Ethyl-acetate. The reaction mixture was mixed with 0.5 kg Hyflo® Super Cel® and filtered two times on a filter layer having pore size 5 um until the filtrate became clear. The filtered crude solid was dissolved in 20 L Acetone at 30° C. The undissolved solid material filtered out and washed with 6 L acetone. The combined filtrate was evaporated to ⅕ volume at reduced pressure and precipitated by the addition of 12 L pre-cooled water (4° C.). Precipitated solid material was filtered out and dried in vacuum at 40° C. to get 426 grams (Yield: 44%) ethyl 2-(4-((tert-butoxycarbonyl)amino)phenyl)-1,4,5,6-tetrahydropyridine-3-carboxylate (Compound 5A) with 99.23 Area % HPLC purity.

1 kg Na₂SO₄ and 5 kg Hyflo® Super Cel® were added to the clear filtrate of the reaction mixture and extracted two times with 75-75 L (2×0.75 vol eq) EtOAc. The combined organic layers were dried on Na₂SO₄, filtered and evaporated to dryness at reduced pressure in order to get 450 grams (Yield: 46%) ethyl (2R,3S)-2-(4-((tert-butoxycarbonyl)amino)phenyl)piperidine-3-carboxylate (Compound 5) with 97.09 Area % HPLC purity and 99.53% ee.

### Example 37: Biocatalytic reduction of ethyl 2-(4-((tert-butoxycarbonyl)amino)phenyl)-1,4,5,6-tetrahydropyridine-3-carboxylate (Compound 5A) Using Imine Reductase Enzyme

Cofactor solution: β-Nicotinamide adenine dinucleotide phosphate disodium salt (NADP+Na, 0.767 grams, 1.00 mmol), glucose (10.40 grams, 57.73 mmol) and glucose dehydrogenase (GDH) enzyme (0.20 grams, GDH(002) purchased from Prozomix Ltd.) were dissolved in 1500 ml 0.1 M potassium phosphate buffer (potassium phosphate buffer pre-prepared by dissolving 35.81 grams of KH2PO4 (0.5 mol) in 2500 ml deionized (DI) water and the pH was adjusted to 7.0 with 5 M KOH solution).

Enzyme solution: Imine reductase enzyme (PRO-IRED-351, 8.0 grams) was dissolved in 200 ml 0.1 M potassium phosphate buffer.

The enzyme solution was diluted with 200 ml of 0.1 M potassium phosphate buffer, and then the cofactor solution was added. A solution of ethyl 2-(4-((tert-butoxycarbonyl)amino)phenyl)-1,4,5,6-tetrahydropyridine-3-carboxylate (Compound 5A, 4.00 grams, 11.55 mmol in 100 ml DMSO) was added, the obtained reaction mixture was stirred at 30° C. for 72 hours.

25 grams of Hyflo® Super Cel® was added to the reaction mixture and then stirred at room temperature for 15 minutes. The filter aid was filtered off on glass filter then was washed with 500 ml of 0.1 M potassium phosphate buffer and DMSO 95:5 ratio mixture.

10.0 grams of Na₂SO₄, 25.0 grams of Hyflo® Super Cel® and 1000 ml of EtOAc were added to the filtrate, then mixed at room temperature for 30 minutes. The filter aids were filtered off, then the solid was washed with additional 1000 ml EtOAc. The phases were separated, the aqueous phase was extracted with two times 2000 ml EtOAc. The EtOAc used for the workup step was previously purged with nitrogen, and the extractions were carried out under nitrogen atmosphere too. The combined organic phases were washed with 6000 ml saturated NaHCO₃ solution, the organic phase was dried on Na₂SO₄, filtered, then evaporated in vacuum.

3.11 grams (66%) of (2R,3S)-2-(4-((tert-butoxycarbonyl)amino)-phenyl)piperidine-3-carboxylate (Compound 5) was isolated as thick yellowish syrup with 97.35 Area % HPLC purity and 98.30% ee, >99.9% de.

### Example 38: Recovery of unreacted ethyl 2-(4-((tert-butoxycarbonyl)amino)phenyl)-1,4,5,6-tetrahydropyridine-3-carboxylate (Compound 5A) from Biocatalytic Reaction Mixture

The filter cake of the biocatalytic reaction mixture was washed with 200 ml of 0.1 M potassium phosphate buffer and DMSO 95:5 ratio mixture. The filtrate was discarded, the wet solid (72.6 grams) was suspended in 726 ml of acetone and stirred at 30° C. for 30 minutes, then filtered off and washed with additional 183 ml acetone.

The combined acetonic filtrates were concentrated in vacuum to ¼th in volume. The recovered substrate was precipitated by adding 300 ml of 4° C. DI water, then let it sit for overnight. The precipitate was then filtered off and dried in vacuum at 40° C. overnight.

0.99 grams (25%) of 2-(4-((tert-butoxycarbonyl)amino)phenyl)-1,4,5,6-tetrahydropyridine-3-carboxylate (Compound 5A) was recovered in a form of white solid with 99.69 Area % HPLC purity.

### Example 39

### 1. Source and Sequence of the Enzymes

The Imine reductase enzyme used as the main enzyme in the preparation of the Compound 5 and Compound 5a were either purchased (for example, PROZOMIX PRO-IRED-128) or prepared (TPW-T164 was prepared as described herein below).

The alcohol dehydrogenase (from Lactobacillus brevis) used a second enzyme for cofactor regeneration (Nicotinamide ADP), was LbADH, TPW-T004, Uniprot: Q03TF9; which was prepared as described herein below.

### 2. Microbial Production and Isolation of the Applied Enzymes

a. Description of Host Strains
TPW-T164 enzyme and TPW-T004 enzyme were produced in Escherichia coli and detailed in Table 1 and Table 2.

TABLE 1
Description of Host Strain - TPW-T164

| | | Expression |
|---|---|---|
| Host Strain | Plasmid | system |
| Escherichia coli | pET28a | IPTG inducible |
| BL21(DE3) | | T7 promoter |

TABLE 2
Description of Host Strain - TPW-T004

| | | Expression |
|---|---|---|
| Host Strain | Plasmid | system |
| Escherichia coli | pMK | Arabinose |
| TOP10 | | inducible |
| | | pBAD promoter |

b. Summary of Enzyme Production

TPW-T004 (LbADH) was produced as intracellular protein under control of pBAD promoter. The seed material was generated in two steps starting from independent, freshly transformed colony. The strain was grown using fed-batch fermentation process. Enzyme expression was induced by addition of Arabinose to a final concentration 1.0%.

TPW-T164 (PRO-IRED-128) was produced as intracellular protein under control of T7 promoter. The seed material was generated in one step starting from aqueous suspension of transformed cells. The strain is grown using fed-batch fermentation process. Enzyme expression is induced by addition of IPTG to a final concentration 0.0024%.

After fermentation, the cells are harvested and collected by separation on a disk stack centrifuge at 4-8° C. Then, cells were resuspended in 100 mM phosphate buffer, cooled to 4-8° C. and mechanically disrupted by homogenization. After that, PEI (Polyethyleneimine) treatment was performed in presence of 60 mM Na₂SO₄ or 60 mM MgSO4. The lysate was incubated in presence of PEI for 30 minutes at RT. Separation of the flocculated cell residue from the liquid was carried out by centrifugation at 4-8° C. for 60 minutes and 3500 RPM. The applied amount of PEI was increased only to an extent until the supernatant after centrifugation became clear. The resulting clear supernatant, containing the enzyme, was cooled to 5° C. and concentrated using 30 kDa ultrafiltration membrane to remove salts and decrease volume. Finally, the concentrated and purified enzyme was lyophilized until water content by TG was NMT 7 w %.

1. A process for kinetic resolution of a racemic amine, which is a racemic 2,3-disubstituted piperidine, by enzymatic oxidation to an imine or to an enamine or their mixture, comprising reacting the racemic 2,3-disubstituted piperidine with an imine reductase enzyme to form a mixture comprising enriched enantiomer of the amine, an imine and/or enamine.

2. The process according to claim 1, wherein the kinetic resolution comprises oxidation of one isomer of the amine to form an imine and/or enamine and wherein one enantiomer of the amine is enriched.

3. The process according to claim 1, wherein the kinetic resolution comprises:
(a) reacting a racemic amine having the formula: 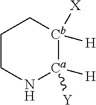
wherein X is alkyl, aryl or functional group, wherein the functional group is an electron withdrawing group, and Y is H, C₁ to C₈ alkyl group, or aryl group, a substituted phenyl, optionally wherein the substituent is selected from 4-amino, 4-t-butoxycarbonylamino, and 4-cyclopentylamino;
wherein one or both of C^{a} and C^{b} is a chiral center, to form a mixture comprising the chiral 2,3-disubstituted piperidine, imine, and enamine: 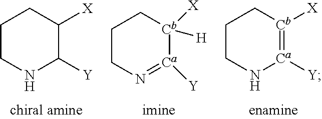
(b) optionally isolating the chiral amine; and
(c) optionally isolating the imine and/or enamine and converting the imine or enamine to the racemic amine.

4. The process according to claim 3, wherein the chiral amine is isolated.

5. The process according to claim 3, wherein X and Y are in a cis configuration.

6. The process according to claim 1, wherein the racemic amine has the formula: 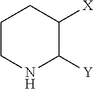
wherein
X is C₁ to C₈ alkyl, C₆ to C₁₀ aryl or functional group, an electron withdrawing group, particularly (C₁ to C₁₀ alkoxy)carbonyl, carboxyl, carbamoyl, cyano, formyl, C₁ to C₈ acyl, (C₁ to C₁₀ alkoxy)carbonyl; and
Y is H, C₁ to C₈ alkyl, or an optionally substituted C₆ to C₁₀ aryl, a substituted phenyl, optionally wherein the substituent is selected from 4-amino, 4-t-butoxycarbonylamino, and 4-cyclopentylamino, particularly wherein Y is 4-t-butoxycarbonylaminophenyl.

7. The process according to claim 1, wherein the racemic amine has the formula: 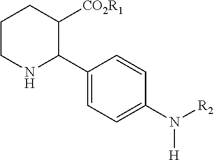
wherein
R₁ is a C₁ to C₈ alkyl group or a C₁ to C₆ alkyl group; and
R₂ is H, C₁ to C₆ alkyl group, a C₅ to C₈ cycloalkyl group, or a protecting group, particularly wherein R₂ is cyclopentyl or t-butyloxycarbonyl (Boc), and more particularly Boc.

8. The process according to claim 7, wherein R₁ is a C₁ to C₃-alkyl group, and wherein R₁ is ethyl or wherein R₁ is ethyl and R₂ is Boc.

9. The process according to claim 1, wherein the reaction is carried out in the presence of a cofactor regenerating system containing NADPH, NADP+ or their mixture as cofactor, optionally wherein the cofactor regenerating system comprises: cofactor regenerating enzyme including at least one of an NADPH oxidase (NOX), an alcohol dehydrogenase (ADH) or a ketoreductase (KRED), and wherein when the cofactor regenerating system comprises ADH or KRED acetone is used as sacrificial substrate and ADH is from Lactobacillus brevis.

10. The process according to claim 1, wherein the imine reductase enzyme has an amino acid sequence that comprises at least 80%, at least 85%, at least 90%, at least 92%, at least 95%, at least 96%, at least 97%, at least 98%, and at least 99% identity to SEQ ID 1:
SEQ ID 1
MTDVSLIGLGPMGMALARALQSSKFTLTVWNRTAERAKPVLNPGTVLAPT
ALAAVQASPVVLVCVADYPASRAILTAPGVHDALRGKVLVQLSTGTPQDA
RDDWAALSGVAYLDGALLATPGQIGRPDTPLFISGEARALAACRPLLEAI
AGNIQHMGEPIGNAAAWDLATLSCMFGAMSGFFHGVRICESEGLGVDAFS
QMIGAISPVLGEMISAEGEAIHANRYGEPESSMATCAGSGRLFVKQAREA
KLDASFPDFLMGLFERSLSAGFANERLAAMVKVMR

11. The process according to claim 1, wherein the imine reductase enzyme has an amino acid sequence that comprises at least 80%, at least 85%, at least 90%, at least 92%, at least 95%, at least 96%, at least 97%, at least 98%, and at least 99% identity to: at least 50, at least 100, at least 150, at least 200, or at least 250, contiguous amino acid residues of SEQ ID 1.

12. The process according to claim 1, wherein the imine reductase enzyme has an amino acid sequence according to SEQ ID 1 which comprises: one or more, five or more, ten or more, 15 or more, 20 or more, 25 or more, 30 or more, 35 or more, or 40, mutations at a position selected from 14, 21, 22, 25, 35, 38, 40, 43, 50, 69, 71, 73, 76, 81, 102, 108, 122, 137, 146, 150, 156, 191, 195, 197, 198, 200, 215, 224, 225, 251, 255, 262, 263, 265, 267, 269, 272, 273, 281, and 284.

13. The process according to claim 1, wherein the imine reductase enzyme has an amino acid sequence corresponding to: SEQ ID 1, SEQ ID 2, SEQ ID 3, or SEQ ID 4.

14. The process according to claim 1, further comprising ex situ recycling of the imine and/or enamine back to the racemic amine, wherein the imine and/or enamine is isolated and converted to the racemic amine.

15. The process according to claim 14, wherein the imine and/or enamine is isolated by extraction, optionally in the presence of an acid or base, by precipitation and filtering, or by filtering from the reaction mixture, and optionally crystallising.

16. The process according to claim 14, wherein the conversion of the imine and/or enamine to the racemic amine is carried out by catalytic hydrogenation using a catalyst, wherein the catalyst is palladium on carbon, platinum (II) oxide, or wherein the catalyst Raney nickel and an organic acid.

17. The process according to claim 1, further comprising dynamic kinetic enzymatic reduction of the imine and/or enamine to form the chiral amine, wherein the dynamic kinetic enzymatic reduction is carried out using an imine reductase enzyme.

18. The process according to claim 17, wherein the reaction is carried out in the presence of a cofactor regenerating system, containing NADPH, NADP+ or their mixture as cofactor, optionally wherein the cofactor regenerating enzyme is glucose dehydrogenase (GDH).

19. The process according to claim 17, wherein the enzyme has an amino acid sequence that comprises at least 80%, at least 85%, at least 90%, at least 92%, at least 95%, at least 96%, at least 97%, at least 98%, and at least 99% identity to SEQ ID 5.

20. The process according to claim 17, wherein the imine reductase enzyme has an amino acid sequence that comprises at least 80%, at least 85%, at least 90%, at least 92%, at least 95%, at least 96%, at least 97%, at least 98%, and at least 99% identity to: at least 50, at least 100, at least 150, at least 200, or at least 250, contiguous amino acid residues of SEQ ID 5.

21. The process according to claim 17, wherein the imine reductase enzyme has an amino acid sequence corresponding to SEQ ID 5 or SEQ ID 6.

22. The process according to claim 17, further comprising:
synthesizing Avacopan, wherein the racemic amine has the formula: 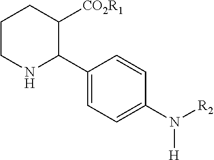
wherein
R₁ is a C₁ to C₈ alkyl group or a C₁ to C₆ alkyl group; and
R₂ is H, a C₅ to C₈ cycloalkyl group, or a protecting group, particularly wherein R₂ is cyclopentyl or t-butyloxycarbonyl (Boc), and more particularly Boc.

23. The process according to claim 22, wherein the racemic amine is Compound 4 having the formula:

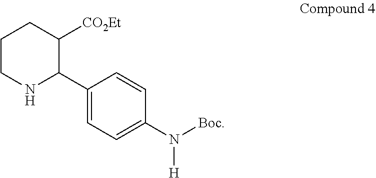 [SMILES: CCOC(=O)C1CCCNC1c1ccc(NC(=O)OC(C)(C)C)cc1]

24. (canceled)

25. (canceled)

26. The process according to claim 22, wherein a resulting enamine Compound 5A having the formula: 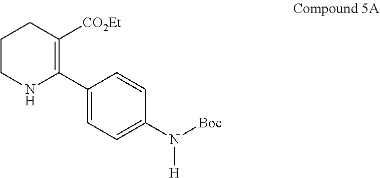 [SMILES: CCOC(=O)C1=C(c2ccc(NC(=O)OC(C)(C)C)cc2)NCCC1]
is isolated optionally by extraction, optionally in the presence of an acid or base, or by precipitation and filtration, crystallized, and converted to the racemic amine.

27. (canceled)

28. (canceled)

29. (canceled)

30. A process for preparing Avacopan, comprising kinetic resolution of cis-ethyl 2-(4-((tert-butoxycarbonyl)amino)phenyl)piperidine-3-carboxylate (Compound 4): 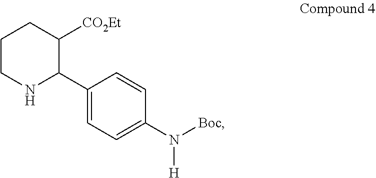 [SMILES: CCOC(=O)C1CCCNC1c1ccc(NC(=O)OC(C)(C)C)cc1]
to form a mixture comprising: ethyl (2R,3S)-2-(4-((tert-butoxycarbonyl)amino)phenyl)-piperidine-3-carboxylate (Compound 5) and ethyl 2-(4-((tert-butoxycarbonyl)amino)-phenyl)-1,4,5,6-tetrahydropyridine-3-carboxylate (Compound 5A): 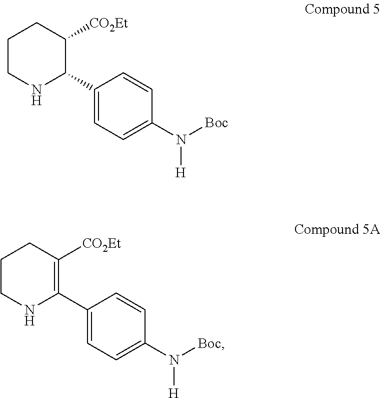 [SMILES: CCOC(=O)[C@H]1CCCN[C@H]1c1ccc(NC(=O)OC(C)(C)C)cc1 ; CCOC(=O)C1=C(c2ccc(NC(=O)OC(C)(C)C)cc2)NCCC1]
optionally using a cofactor regenerating enzyme, particularly selected from NADPH oxidase (NOX), alcohol dehydrogenase (ADH) or ketoreductase (KRED), wherein the kinetic resolution of the cyclic amine compound is carried out by an imine reductase enzyme and ADH cofactor regenerating system.

31. The process according to claim 30, wherein the cofactor regenerating enzyme is ADH or KRED and the kinetic resolution is carried out using a catalytic amount of nicotinamide adenine dinucleotide phosphate (NADP+) cofactor and acetone as sacrificial substrate.

32. The process according to claim 30, comprising recycling Compound 5A via extraction at acidic pH or by precipitation and filtering, or by filtering from the reaction mixture, and optionally crystallising.

33. The process according to claim 30, wherein Compound 5A is isolated by acid-base extraction, precipitation and filtering, or by filtering from reaction mixture, and optionally crystallising, thereby providing a solution comprising Compound 5, wherein Compound 5 is converted to Avacopan, and optionally recycling the filtered Compound 5A to Compound 4.

34. The process according to claim 30, comprising crystallizing Compound 5A from the reaction mixture comprising Compound 5 and Compound 5A, isolating the crystallized Compound 5A and further converting it to Compound 4, and converting Compound 5 to Avacopan.

35. The process according to claim 30, wherein Compound 5A is converted back to Compound 4, by hydrogenation using a catalyst including at least one of Pd/C, Pt₂O, or Raney nickel in acetic acid.

36. The process according to claim 30, wherein Compound 5A is converted to Compound 5 by dynamic kinetic reduction-using an imine reductase enzyme and glucose dehydrogenase (GDH) cofactor regenerating enzyme.

37. The process according to claim 30 wherein the kinetic resolution of the cyclic amine is carried out using an imine reductase enzyme having an amino acid sequence corresponding to SEQ ID 5 or SEQ ID6.

38. The process according to claim 36, wherein the Compound 5A is converted to Compound 5 by dynamic kinetic enzymatic reduction using an imine reductase enzyme having an amino acid sequence that comprises at least 80%, at least 85%, at least 90%, at least 92%, at least 95%, at least 96%, at least 97%, at least 98%, and at least 99% identity to: at least 50, at least 100, at least 150, at least 200, or at least 250, contiguous amino acid residues of SEQ ID 5.

39-98. (canceled)
